(19) United States
(12) Patent Application Publication
(10) Pub. No.: US 2023/0094545 A1
(43) Pub. Date: Mar. 30, 2023

(54) S1P RECEPTOR MODULATORS

(71) Applicant: AKAAL PHARMA PTY LTD, Bundoora (AU)

(72) Inventors: DAMIAN GROBELNY, Bundoora (AU); GURMIT SINGH GILL, Bundoora (AU)

(73) Assignee: AKAAL PHARMA PTY LTD, Bundoora (AU)

(21) Appl. No.: 17/799,904

(22) PCT Filed: Feb. 26, 2021
(86) PCT No.: PCT/AU2021/050169

§ 371 Date: Aug. 15, 2022

(30) Foreign Application Priority Data
Feb. 28, 2020 (AU) 2020900580

(51) Int. Cl. A61K 31/4245; A61P 9/00; A61P 17/04; A61P 29/00; A61P 25/02; A61P 25/28; A61P 1/04; A61P 19/02; A61P 17/06; A61K 9/00; A61P 37/08; A61K 9/20; A61K 9/06; A61K 9/08; A61K 9/70; A61K 31/455; A61K 31/355; A61K 31/573; A61K 31/196; A61P 17/02; A61P 17/10; A61K 45/06; A61K 47/10; A61K 47/06; A61K 47/12; A61K 47/14; A61K 47/44; A61K 47/22; A61K 47/38

(52) U.S. Cl. CPC A61K 31/4245; A61P 9/00; A61P 17/04; A61P 29/00; A61P 25/02; A61P 25/28; A61P 1/04; A61P 19/02; A61P 17/06; A61K 9/0014; A61P 37/08; A61K 9/0053; A61K 9/2018; A61K 9/2059; A61K 9/2054; A61K 9/2027; A61K 9/2009; A61K 9/2013; A61K 9/06; A61K 9/08; A61K 9/7023; A61K 31/455; A61K 31/355; A61K 31/573; A61K 31/196; A61P 17/02; A61P 17/10; A61K 45/06; A61K 47/10; A61K 47/06; A61K 47/12; A61K 47/14; A61K 47/44; A61K 47/22; A61K 47/38

(57) ABSTRACT

The current invention is based on the determination that a S1P receptor modulator compound of formula (I):

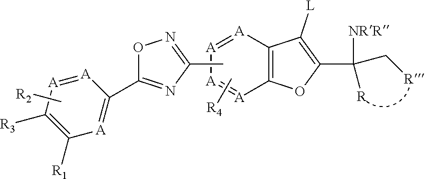

decreases the heart rate of a subject to which it is administered by about 5 beats/min or less daily, or about 4 beats/min or less daily, or about 3 beats/min or less daily, or about 2 beats/min or less daily, wherein the S1P receptor modulator is administered at an initial daily dosage which is substantially the same as the standard daily therapeutic dosage.

### FIELD

This disclosure relates to methods of treating or preventing diseases or disorders, particularly inflammation, immune mediated disorders, and vascular and neuronal disorders. The methods include administering medicaments containing S1P receptor modulators which result in the minimisation of bradycardia and lymphopenia.

### BACKGROUND

### S1P Receptor Axis and Relevance to Disease Inflammation

S1P receptors are a family of G-protein-coupled receptors with a wide range of expression over major organ systems such as immune, nervous and vascular systems. There are five receptors known as Sphingosine 1-phosphate receptors (S1P1-5), with the common endogenous ligand S1P having a variety of downstream effects (Cooke et al, Annual Reports in Medicinal Chemistry, 2007, 42, pp 245-263, and references therein). The S1P receptors, especially the type 1 receptor S1P1, are involved in the immune response, endothelial barrier enhancement, (Wilkerson B A et al, J Biol Chem, 2012, Vol. 287, 44645) cellular protection (Rutherford C et al, Cell Death and Disease, 2013, 4, e927; doi:10.1038/cddis, 455), cell differentiation, cell mobilization/chemotaxis and others.

The downstream effect through the S1P receptor is known to involve the mTOR modulation and immune modulation (Liu G. et al, Nat Immunol, 2010, 11, 1047). S1P receptor involvement is well documented in the inhibition of the STAT3 (Garris C. S. et al, Nat Immunol, 2013, Vol 14, 1166) which is a known target involved in inflammations and cancer. The S1P receptors are well known to modulate pain (Welch S. P. et al, Biochem Pharmacol, 2012, 84, 1551). Further, S1P receptors are involved in stem cell chemotaxis (Kimura A. et al, Stem Cell, 2007, 25, 115) and regeneration (Leronimakis et al. Skeletal Muscle, 2013, 3, 20) and the S1P axis is involved in neuroprotection (Asle R M et al, EXCLI Journal, 2013, 12, 449). S1P receptor modulation is involved in the expression of cytokines such as TNF_{α}, IL6, IL12, VEGF (Bolick D T et al, Arterioscler Thromb Vasc Biol, 2005, 25, 976; Sanchez T, et al, J Biol Chem 2003, 278 (47), 47281). S1P receptors have shown major involvement in critical illnesses such as acute lung injury, influenza and others. The endothelial cells make the inner layer of blood vessels express the S1P receptors and S1P1 agonists are well known to enhance the vascular barrier and prevent vascular leakage and enhance vasculature maturation (McVerry B. J. et al, J Cell Biochem, 2004, 92, 1075; Allende M. L. et al, Blood, 2003, 102, 3665; Paik J. et al, Genes Dev, 2004, 18, 2392; Garcia J. G. N. et al, J Clin Invest, 2001, 108(5), 689). The S1P receptor axis is involved in inflammations and cancer (Kunkel G. T. et al, Drug Discovery, 2013, 12, 688).

Inflammation is an immune response to injury and infection. Symptoms include redness, heat, swelling and pain. The control of inflammation is important in regeneration and wound healing, however uncontrolled inflammation may give rise to a prolonged and damaging response resulting in chronic disease. Inflammation may be local or organ specific or it may spread over the body giving rise to systemic disease.

An inflammatory site has overexpressed pro-inflammatory cytokines and factors such as interleukins (IL1, IL6, IL17), tumour necrosis factor (TNFα), inducible-nitroxide-synthase (iNOS), cyclooxygenase-2 (COX-2), myeloperoxidase (MPO) and vascular endothelial growth factor (VEGF). These cytokines and factors are involved in the recruitment of immune cells, altered immune response, endothelial barrier disruption, altered differentiation and aberrant proliferation patterns within the inflammation. The blood vessels become abnormal, dilated, leaky, tortuous and there is fluid accumulation and edema. The remote parts are devoid of blood supply and develop hypoxia with possible onset of cancer. There is an altered pattern of cell survival and degeneration occurs.

An increasing body of scientific data reveals that inflammation is involved in nearly all ailments. Multiple diseases of inflammatory origin are common in various body organ systems such as cardiovascular (i.e. atherosclerosis, ischemic diseases, venous disease) nervous system (i.e. multiple sclerosis, epilepsy, ALS, neuropathy) and immune mediated diseases (i.e. rheumatoid arthritis, asthma, psoriasis, atopic dermatitis, acne, vitiligo). The inflammations have an underlying role in ischemic injury, atherosclerotic lesions (Galkina E. et al, Annu Rev Immunol, 2009, 27, 165) and cancer tumours (Landskron G. et al, J Immunol Res, 2014, Article ID 149185, 19 pages http://dx.doi.org/10.1155/2014/149185).

Diseases such as rheumatoid arthritis, joint pain, muscle inflammation, psoriasis, dermatitis, uveitis, and atherosclerosis are accompanied by inflammation, along with other co-pathologies such as pain, itching and degeneration. S1P receptors are known targets for multiple pathologies occurring in inflammatory indications; particularly the S1P1 receptor axis in inflammation and immune modulation.

Cancer of various origins has common pathologies such as inflammation, vascular abnormalities (leaky vessels, neo angiogenesis), hypoxia, aberrant differentiation, extravasation of cells from the primary place of cancer and metastasis. S1P receptor modulation may alleviate the multiple pathologies found in various cancers in a single treatment by alleviating inflammation, barrier enhancement, avoiding metastasis and cell differentiation. S1P receptor mediated cell clamping is reported to augment cell-cell adhesion thereby blocking tumour cell intravasation from the point of cancer (Feng H, Cancer Cell, 2010, 18(4), 353-366).

Vascular diseases such as aberrant blood vessels, leaky and fluid extravasation and edema hyper vascularity are also thought to be a result of underlying inflammation. Neurodegeneration, inflammation and vascular leak, and hyper vascularity are common in macular degeneration, glaucoma, retinopathy. Lung inflammation is a central reason for various pulmonary problems such as asthma, chronic obstructive pulmonary disease (COPD), acute lung injury and influenza.

Myocardial infraction, spinal injuries and ischemic injuries are related to inflammation, cell death and compromised function of critical organs. S1P receptor modulation can alleviate the pathologies by halting inflammation, rescuing the cell death, (Schabbauer G. et al, Arterioscler Thromb Vasc Biol, 2004, 24, 1963; Wang J. et al., Biomaterials, 2015, 62, 76), improving blood flow, attracting stem cells to the site of injury, differentiation and regeneration (Leronimakis et al, Skeletal Muscle, 2013, 3, 20). The use of S1P receptor modulators can be extended to wound healing and regeneration of muscle, bone and other organs including transplant success (Lia L et al, Cornea, 2014, 33 (4), 398).

S1P receptor modulation can address multiple pathological events (FIG. 1) common in various diseases of humans, animals and other species. However, its promise as a key drug target is hampered by the appearance of significant clinical side effects such as lymphopenia and bradycardia. There is no causal link between lymphopenia and positive treatment outcomes in S1P receptor modulator therapy.

Many multiple sclerosis disease-modifying therapies result in a corresponding decrease in circulating T and B lymphocytes. However, the degree of lymphopenia in peripheral blood was not associated to the positive treatment outcome of FTY720 in relapsing remitting multiple sclerosis (RRMS) patients (Fragoso Y. D. et al, Multiple Sclerosis and Related Disorders, 2018, 19, 105-108 and references therein).

Another study in an animal model of multiple sclerosis (MS) indicated the lack of association of lymphopenia with treatment outcomes. As soon as dosing with FTY720 was discontinued the severity of lymphopenia was sustained while the disease score increased. Following discontinuation of drug treatment, clinical signs reappeared after a few days in mice, which relapsed to similar disease scores seen in untreated controls (M. Webb et al., Journal of Neuroimmunology, 2004, 153, pp 108-121). Thus, the higher level of lymphopenia observed after treatment of autoimmune diseases such as multiple sclerosis with S1P modulators is irrelevant and appears to be a non-desired side effect.

Lymphopenia leads to alteration of the immune system and contributes to unwanted adverse effects (Pierre-Eric Juifa P-E. et al, Expert opinion on drug metabolism & toxicology, 2016, 12, (8), 879-895). In turn this can result in the discontinuation of S1P drug therapy or treatment breaks (Johnson T A, Clinical Immunology, 2010, 137, 15-20), which then elevates the risk of disease symptom recurrence or rebound of disease activity (Joachim B. Havla et al, Arch Neurol, February 2012, Vol 69, No. 2).

Instead, recent studies suggest that it is the direct effects of S1P1 receptor modulation and/or agonism of S1P receptors in a variety of cells and organs that appear to be relevant to therapeutic efficacy, rather than the lymphocyte cells or immune cells.

In an animal model of kidney disease, while disease symptoms were significantly reduced with both FTY720 and SEW2871 S1P1 modulators, only FTY720 was associated with reduced total lymphocyte levels, suggesting that the effect was independent of the lymphopenia (Awad A et al, Kidney Int., 2011, 79(10), 1090-1098). In another animal model of acute kidney disease (AKI), the activation of endothelial S1P1 was found to be necessary to protect from IRI and permit recovery from AKI (Perry H M et al, Am Soc Nephrol, 2016, 27, 3383-3393). Similarly, in an animal model of neuropathy, it is the direct effect of S1P1 modulation on astrocytes which is relevant for efficacy in neuropathy. The cellular and molecular mechanisms engaged by the S1PR1 axis in neuropathic pain establish S1PR₁ as a key target for therapeutic intervention with S1PR1 modulation as a class of nonnarcotic analgesics. (Chen Z et al, PNAS, 2019, vol. 116, no. 21, 10557-10562).

In another study, the direct effect of S1P1 modulation on astrocytes was relevant for efficacy in multiple sclerosis. The data identify non-immunological CNS mechanisms of FTY720 efficacy and implicate S1P signalling pathways within the CNS as targets for multiple sclerosis therapies (Choi J W et al, PNAS, 2011, vol. 108, no. 2, 751-756). The S1P1 receptor activity is related to myelination process and MS disease alleviation by FTY720 (Sheridan G K et al. GLIA, 2012, 60:382-392 Cho J W et al. PNAS, 2011, 108, 2, 751). A local dose of FTY720 achieves the opposite and promotes demyelination and the drug is not successful in primary progressive multiple sclerosis, which is a more neurodegenerative form of MS (Hu Y et al, Molecular and Cellular Neuroscience, 2011, 48, 72-81; Lublin F et al, Lancet, 2016, 387, 1075-84) and this adverse effect may be attributed to S1P1 potent receptor degradation by FTY720.

The direct effect on endothelial cells was relevant to efficacy in an animal model of influenza. Cytokine storm during viral infection is a prospective predictor of morbidity and mortality, yet the cellular sources remain undefined. S1P1 receptors are expressed on endothelial cells and lymphocytes within lung tissue, and the study showed that S1P₁ modulation of these cells suppressed cytokine release and innate immune cell recruitment in wild-type and lymphocyte-deficient mice, identifying endothelial cells as central regulators of cytokine storm (Teijaro J W et al, Cell, 2011, 146, 980-991).

S1P1 receptor suppression of cytokine storms may also have value in viral indications. S1P modulator therapy alone has been shown to have greater clinical efficacy compared to antiviral therapy alone against the pathogenic influenza virus in mice (82% vs 50% mortality). Additionally, the combination of both S1P1 modulation and antiviral therapy resulted in a near complete protection from mortality (96%) (Walsh K B, PNAS, 2011, 12018-12023).

S1P1 receptor modulation is also involved in bone regeneration, (Yang-Hee Kim et al, Biomaterials, 2014, 35 (1), 214-224) muscle healing (Nicholas Ieronimakis et al, Ieronimakis et al. Skeletal Muscle 2013, 3:20), neuronal regeneration (Safarian et al, J Mol Neurosci, 2015, 56, 177), pain alleviation (Stockstill et al, J Pain, 2014, 15(4), S60).

S1P1 modulation is also desired in multiple cardiovascular and neuronal diseases due to its ability to exert direct effects on endothelial and/or neuronal cells, however, lymphopenia is again an undesirable side effect which plays no role in the treatment of these indications and instead hampers continuous therapy.

Accordingly, there is a need for improved 51P1 modulators which only cause a low level of lymphopenia and can therefore be used safely in, for example, immune mediated and/or cardiovascular and/or neuronal indications.

Bradycardia is another common and significant side effect in current S1P therapies. Human clinical trials assessing S1P1 modulators, including the drug FTY720, frequently report the induction of 1st dose bradycardia (Bigaud M. et all, Biochimica et Biophysica Acta 2014, 1841, 745-758 and references therein; Pierre Eric J et al, Expert Opinion on Drug Metabolism & Toxicology, 2016, VOL. 12, NO. 8, 879-895; Pierre Eric J et al, Int. J. Mol. Sci. 2017, 18, 2636; Tran J Q et al, The Journal of Clinical Pharmacology, 2017, 00(0), 1-9; US 2019/0091180 A1; Siponimod: USOO8492441 B2, July 2013; Abstract Presented at the 13th Congress of the European Crohn's and Colitis Organisation (ECCO), Feb. 14-17, 2018, Vienna, Austria).

Accordingly, a dose titration strategy is typically required to mitigate or reduce potential side effects, wherein the initial days of dosing involves a low sub-therapeutic dose level and after several days the dose level is steadily increased to a standard daily therapeutic dose. This dose titration strategy however results in a loss of total treatment days at therapeutic dose levels.

The observance of bradycardia following S1P receptor therapy may prohibit their use in subjects who are susceptible to heart failure, arrhythmias, high grade atrio-ventricular blocks, sick sinus syndrome, history of Syncopal episodes and those on anti-arrhythmic treatment. Moreover, for the subject with acute indication and in urgent need of a dose of therapeutic level, such as in critical care, sepsis, stroke, or organ injury, dose titration is not a valid option.

While bradycardia and lymphopenia are evident in S1P1 modulator therapy, these two events are independent and not linked, as proven in studies utilising the clinical compound GSK2018682. In single dose sessions, 2 mg or lower doses of GSK2018682 induced minimal or no lymphopenia beyond the range of normal daily circadian variation (up to approximately 20%); however, bradycardia was evident at this dose level with reduction in heart rate of 10 beats or more. (Xu J et al, Clinical Pharmacology in Drug Development, 2014, 3(3) 170-178).

Furthermore, the observance of bradycardia with S1P1 selective agonists has been reported to be species specific. S1P1 receptor selective agonists do not induce bradycardia in rodents, however, the S1P1 selective agonists tested in humans resulted in significant bradycardia in patients (Juif P-E. et al, Int. J. Mol. Sci. 2017, 18, 2636; doi:10.3390/ijms18122636; Pali L. et al, Pharmacol Res Perspect, 2017; e00370. wileyonlinelibrary.com/journal/prp2|1 of 12; Rey M et al, PLOS ONE, September 2013 Volume 8|Issue 9|e74285). In view of this, the behaviour of S1P modulators in humans is unpredictable and results in animal studies are not readily translatable to humans.

Accordingly, there is a need for improved dosage regimens for S1P receptor modulators that do not result in significant side effects, such as bradycardia and/or lymphopenia, even after long-term dosage.

There is a further unmet need to improve dosing and safer use of S1P receptor modulators in the treatment of disease, particularly inflammation, immune mediated disorders, and vascular and neuronal disorders. The present disclosure addresses one or more of these unmet needs.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

### SUMMARY

This disclosure relates to medicaments comprising S1P receptor modulators for the treatment of disease, particularly inflammation, immune mediated disorders, vascular disorders and neuronal disorders and to advantageous dosing regimens for the medicaments. S1P receptor modulators are currently used to treat diseases or disorders involving inflammation or pain, however the administration and dosage regimens associated with such modulators cause significant side effects, such as bradycardia and long-term moderate to high lymphopenia.

Bradycardia, as would be understood by the skilled person, is a slower than normal heart rate. It can vary from person to person as a result of, for example, their age and physical fitness. But a skilled person such as a physician will be readily able to determine when a subject is bradycardic. A normal adult resting heart rate is between 60-100 beats per minute (bpm). In bradycardic subjects of average health and fitness, the heart beats fewer than 60 times a minute.

Lymphopenia, also referred to as lymphocytopenia, occurs when your lymphocyte count in your bloodstream is lower than normal. As would be understood by the skilled person, and adjusting for the age of the subject, a diagnosis of lymphopenia means that your blood lymphocyte count is below 1,500 cells/microliter. Infants and children have more lymphocytes; less than 3,000 cells/microliter is considered to be too low in this case.

While a dose titration strategy is frequently required with prior art S1P receptor modulator therapy, the methods and medicaments comprising S1P receptor modulators disclosed herein advantageously allow treatment to begin immediately at a therapeutic dose level and may also provide other advantages, such as improved clinical safety and patient satisfaction and compliance. Further, the methods and medicaments comprising S1P receptor modulators as disclosed herein mitigate or reduce lymphopenia, thereby avoiding unwanted immune suppression which can pose the threat of opportunistic infections, or cancers.

Moreover, concerns regarding the side effects of prior art S1P receptor modulators or agonists may restrict safer use of S1P modulators for indications other than autoimmune indications, such as vascular or neuronal indications. Prior art S1P receptor modulators or agonists may reduce the cardiac rhythm and induce bradycardia which lasts for several hours post dosing. As a consequence of this side effect, the S1P modulator or agonist therapy may have to be initiated under close medical supervision in order to ensure that the cardiac rhythm is maintained at an acceptable level. This may involve the hospitalization of patients, making treatment more expensive and complex. In the treatment of other indications, topical or other applicable routes of administration of prior art S1P receptor modulators or agonists may be restricted to sub-therapeutic levels due to these side effects (for example, limited area and/or limited dose levels).

According to one aspect of the present disclosure, there is provided a method of treating or preventing a disease or disorder comprising administering to a subject in need thereof a medicament, said medicament comprising a therapeutically effective amount of an S1P receptor modulator, wherein said medicament decreases the heart rate of the subject by about 5 beats/min or less daily, or about 4 beats/min or less daily, or about 3 beats/min or less daily, or about 2 beats/min or less daily, and wherein the S1P receptor modulator is administered at an initial daily dosage which is substantially the same as the standard daily therapeutic dosage.

According to another aspect of the present disclosure, there is provided a therapeutically effective amount of an S1P receptor modulator for use in treating or preventing a disease or disorder wherein said medicament decreases the heart rate of the subject by about 5 beats/min or less daily, or about 4 beats/min or less daily, or about 3 beats/min or less daily, or about 2 beats/min or less daily, and wherein the S1P receptor modulator is administered at an initial daily dosage which is substantially the same as the standard daily therapeutic dosage.

In a further aspect there is provided use of a therapeutically effective amount of an S1P receptor modulator in the preparation of a medicament for treating or preventing a disease or disorder wherein said medicament decreases the heart rate of the subject by about 5 beats/min or less daily, or about 4 beats/min or less daily, or about 3 beats/min or less daily, or about 2 beats/min or less daily, and wherein the S1P receptor modulator is formulated for administration at an initial daily dosage which is substantially the same as the standard daily therapeutic dosage.

Preferably the S1P receptor modulator is a compound of Formula (I) as shown herein, and most preferably is

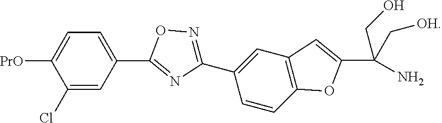

As used herein in each of the aspects and embodiments described throughout, the term ‘initial daily dosage’ refers to the first dosage level at which the S1P receptor modulator is administered to the patient. As used herein the term ‘standard daily therapeutic dosage’ refers to the dosage level which is sufficient to deliver a therapeutically effective amount of SW receptor modulator to the patient.

The standard daily therapeutic dosage may be delivered via injection, orally, topically or via medical device with varied systemic exposure of the patient. The standard daily therapeutic dosage may also refer to the dosage level sufficient to control the symptoms or halt disease progression effectively in a patient. The term ‘substantially the same’ refers to two values being similar or the same, but is understood to encompass a range of normal tolerance in the art. For example, this may be within two standard deviations of the mean dosage.

In some embodiments the difference between the initial daily dosage and the standard daily therapeutic dosage is less than 25%, or less than 15%, or less than 10%, or less than 5%.

In some embodiments the initial daily dosage is the same as the standard daily therapeutic dosage.

Typically, in prior art S1P receptor modulators, the initial daily dosage may be far lower than the standard therapeutic dosage, and may be increased stepwise or only once until the standard therapeutic dosage is reached. Advantageously the present disclosure provides a method whereby the dosage may be started at the standard therapeutic dosage, or slightly less than the standard therapeutic dosage, without the emergence of side effects associated with prior art S1P receptor modulator therapy.

In some embodiments the standard daily therapeutic dosage of S1P receptor modulator is up to 70 mg via oral administration or injection and up to 3 g via topical.

In some embodiments the standard daily therapeutic dosage of S1P receptor modulator is up to 24 mg via oral administration or injection.

In some embodiments the standard daily therapeutic dosage of S1P receptor modulator is between 0.5 mg and 12 mg via oral administration or injection, preferably ≤6 mg daily oral.

In some embodiments the administration of the medicament does not cause a substantial decrease in heart rate.

In some embodiments the administration of the medicament does not cause bradycardia. Accordingly in another aspect of the invention, there is provided a method of preventing or ameliorating the risk of bradycardia in a subject receiving a medicament comprising a therapeutically effective amount of an S1P receptor modulator, wherein said medicament decreases the heart rate of the subject by about 5 beats/min or less daily, or about 4 beats/min or less daily, or about 3 beats/min or less daily, or about 2 beats/min or less daily, and wherein the S1P receptor modulator is administered at an initial daily dosage which is substantially the same as the standard daily therapeutic dosage.

In a further aspect of the invention, there is provided a method of preventing or reducing the level of lymphopenia in a subject receiving a medicament comprising a therapeutically effective amount of an S1P receptor modulator, wherein said medicament decreases the heart rate of the subject by about 5 beats/min or less daily, or about 4 beats/min or less daily, or about 3 beats/min or less daily, or about 2 beats/min or less daily, and wherein the S1P receptor modulator is administered at an initial daily dosage which is substantially the same as the standard daily therapeutic dosage.

In some embodiments the level of lymphopenia is ≤25%.

In some embodiments the level of lymphopenia is ≤50%.

In some embodiments the level of lymphopenia is ≤70%.

Preferably the S1P receptor modulator in the above mentioned aspects and embodiments of the invention is a compound of Formula (I) as shown herein, and most preferably is

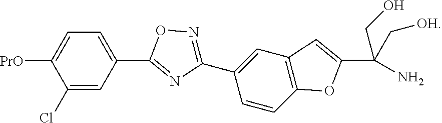

Furthermore the compounds of formula (I) of the invention are effective in treating or preventing pruritis. Pruritis, as would be understood by the skilled person, is itchiness, and particularly itchiness of the skin, which can have a number of causes of particular relevance to this invention, pruritis as a result of psoriasis, dermatitis, prurigo nodularis and other indications. There is therefore provided a method of preventing or treating pruritus, comprising administering a medicament to a subject in need thereof, said medicament comprising an effective amount of an S1P receptor modulator, preferably as a topical medicament. More preferably the S1P receptor modulator is a compound of Formula (I) as shown herein, and most preferably is

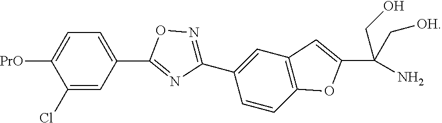

Successful treatment would see the subjects itchiness ameliorated, lessened in severity or eliminated. Prophylactic administration of the medicament comprising an effective amount of an S1P receptor modulator can prevent itching from starting or developing.

Furthermore the compounds of formula (I) of the invention are effective in treating or preventing pain, and in particular, neuropathic pain, arthritis pain and wound pain. Accordingly there is also provided a method of preventing or treating pain, preferably neuropathic pain, muscle pain and wound pain, comprising administering a medicament to a subject in need thereof, said medicament comprising an effective amount of an S1P receptor modulator, preferably as an oral or topical medicament. More preferably the S1P receptor modulator is a compound of Formula (I) as shown herein, and most preferably is

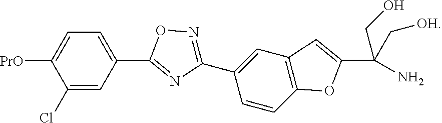

The compounds of formula (I) of the invention have also been determined to be useful in treating acne and rosacea. Acne, also known as acne vulgaris, involves inflammatory papules and pustules on the skin as a result of clogged hair follicles. Rosacea can involve similar, pus filled bumps. The inventors have demonstrated that compounds of formula (I) of the invention are effective in treating acne and/or rosacea. Accordingly, there is also provided a method of treating either or both of acne and rosacea, comprising administering a medicament to a subject in need thereof, said medicament comprising an effective amount of an SW receptor modulator, preferably as a topical medicament. More preferably the S1P receptor modulator is a compound of Formula (I) as shown herein, and most preferably is

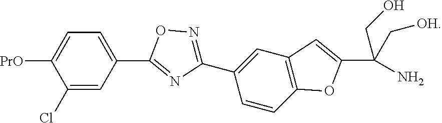

The S1P receptor modulator of Formula (I) as shown herein, and most preferably being

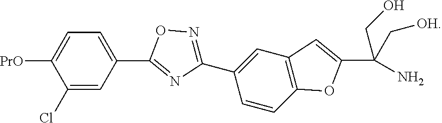

may also be used in a medicament for treating or preventing pruritis, or for treating or preventing pain, or for treating acne and/or rosacea. Similarly, there is provided use of a S1P receptor modulator of a compound of Formula (I) as shown herein, most preferably being

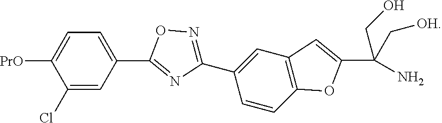

in the preparation of a medicament for treating or preventing pruritis, or a medicament for treating or preventing pain, or a medicament for treating acne and/or rosacea. Preferably the medicament is a topical medicament.

The inventors have also made the surprising determination that the compounds of formula (I) of the invention are able to inhibit mould growth. Accordingly, in an alternative aspect of the invention, there is provided a method of inhibiting or preventing mould growth, comprising administering a composition, said composition comprising an effective amount of an S1P receptor modulator. The composition may be administered to animal or plant subjects where mould growth occurs. In one embodiment of this aspect of the invention, the method treats or prevents a condition or disease caused by the mould that is inhibited by the compounds of the invention. The disease or condition may be of an animal or a plant. The mould may be, for example, Rhizopus stolonifera.

In some embodiments, as mentioned above, the S1P receptor modulator is a compound of formula (I):

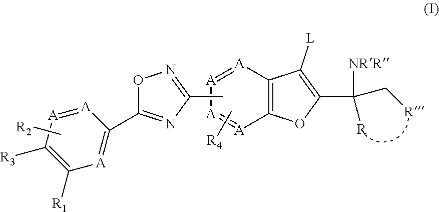

wherein R1 is selected from the group consisting of hydrogen, deuterium, halogen, CN, CF3, —COOH, amide, sulphonamide, alkoxy, aryloxy, nitro, a C1-6 alkyl group, said alkyl group optionally comprising one or more of deuterium, O, S, NR′ (R′═H, alkyl, cycloalkyl), halogen, a carbon-carbon double bond, a carbon-carbon triple bond, a carbon-nitrogen double bond, a carbon-nitrogen triple bond, heterocycle, aryl, alkyl and cycloalkyl (C3-7);

wherein R2 is selected from the group consisting of hydrogen, deuterium, halogen, CN, CF3, nitro, alkoxy, aryloxy, a C1-4 alkyl group, said alkyl group optionally comprising one or more of deuterium, O, S, NR′ (R′═H, alkyl, cycloalkyl), halogen, a carbon-carbon double bond, a carbon-carbon triple bond, a carbon-nitrogen double bond, a carbon-nitrogen triple bond, heterocycle, aryl and C3-7 cycloalkyl;

wherein R3 is selected from the group consisting of hydrogen, deuterium, halogen, alkoxy, aryloxy, a C1-6 alkyl group, said alkyl group optionally comprising one or more of deuterium, O, S, NR′ (R′═H, alkyl, cycloalkyl), halogen, a carbon-carbon double bond, a carbon-carbon triple bond, a carbon-nitrogen double bond, a carbon-nitrogen triple bond, heterocycle, aryl, alkyl and C3-7 cycloalkyl;

wherein R4 is selected from the group consisting of hydrogen, deuterium, halogen, CN, CF3, a C1-4 alkyl group, said alkyl group optionally comprising one or more of deuterium, O, S, NR′ (R′═H, alkyl, cycloalkyl), halogen, a carbon-carbon double bond, a carbon-carbon triple bond, a carbon-nitrogen double bond, a carbon-nitrogen triple bond, heterocycle, aryl, alkyl and C3-7 cycloalkyl;

wherein A, independently in each occurrence, represents a carbon or nitrogen atom with the proviso that a ring has no more than two nitrogen atoms;

wherein L is selected from the group consisting of hydrogen, deuterium, F, Cl, Br and C1-3 alkyl;

wherein R is selected from the group consisting of H, COOH, C1-4 alkyl and C1-4 hydroxy-alkyl;

wherein R′ and R″ are independently selected from H and C1-4 alkyl;

wherein R′″ is selected from OH, —OPO3H2 and physiologically acceptable salts;

wherein [symbol] represents an optional bridging group;

or a pharmaceutically acceptable salt thereof.

In some embodiments R3 is selected from the group consisting of Me, OMe, OEt, OPr, O-iPr, O-isobutyl, O-isopentyl, O-cyclopentyl, O-allyl and O-benzyl.

In some embodiments the S1P receptor modulator is a compound of formula (II)

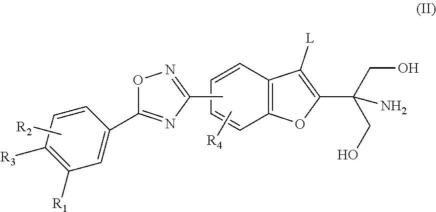

wherein R1 is selected from hydrogen, deuterium, halogen, CN, CF₃, —COOH, amide, sulphonamide, alkoxy, aryloxy, nitro and an alkyl chain (C1-5), said alkyl chain optionally containing one or more of deuterium, O, S, NR′ (R′═H, alkyl, cycloalkyl), halogen, a multiple bond, heterocycle, aryl, cycloalkyl (C3-7) and carbocycle;

wherein R2 is selected from hydrogen, deuterium, halogen, CN, CF₃, an alkyl chain (C1-4) said alkyl chain optionally containing one or more of deuterium, O, S, NR′ (R′═H, alkyl, cycloalkyl), halogen, a multiple bond, heterocycle, aryl, cycloalkyl (C3-7) and carbocycle;

wherein R3 is selected from hydrogen, deuterium, halogen, alkoxy, aryloxy, an alkyl chain (C1-7), said alkyl chain optionally containing one or more of deuterium, O, S, NR′ (R′═H, alkyl, cycloalkyl), halogen, a multiple bond, heterocycle, aryl, cycloalkyl (C3-7) and carbocycle;

wherein R4 is selected from hydrogen, deuterium, halogen, CN, CF₃, an alkyl chain (C1-4), said alkyl chain optionally containing one or more of deuterium, O, S, NR′ (R′═H, alkyl, cycloalkyl), halogen, a multiple bond, heterocycle, aryl and cycloalkyl (C3-7);

wherein L is selected from hydrogen, deuterium, F, Cl, Br and alkyl (C1-3).

In some embodiments the compound of formula (II) has

R1 selected from F, Cl, Br, CN, CF3, NO2, Me, OMe, OEt, OPr, O-iPr, O-isobutyl, O-isopentyl, O-cyclopentyl, O-allyl, O-benzyl and;

R2 selected from H, deuterium, F, Cl, Br, CN, CF3, NO2, Me, OMe, OEt, OPr, O-iPr, O-isobutyl, O-isopentyl, O-cyclopentyl, O-allyl, O-benzyl and;

R3 selected from H, deuterium, Pr, butyl, OMe, OEt, OPr, OiPr, O-isobutyl, O-isopentyl, O-butyl, O-pentyl, O-cyclopentyl, O-allyl, O-benzyl and;

R4 selected from H, deuterium, Me and Et; and

L selected from H, deuterium, Me and Cl.

In some embodiments the compound of formula (II) has

R1 selected from F, Cl, Br, CN, CF3, Me, NO2, OMe, OEt, OPr, O-iPr, O-isobutyl,

O-isopentyl, O-cyclopentyl, O-allyl, O-benzyl and;

R2 is H;
R3 selected from H, deuterium, Pr, butyl, OMe, OEt, OPr, OiPr, O-isobutyl, O-isopentyl, O-butyl, O-pentyl, O-cyclopentyl, O-allyl, O-benzyl and;
R4 selected from H, deuterium, Me and Et; and L is H.

In some embodiments the subject is a mammal or a bird, preferably a human.

In some embodiments the medicament is administered to a subject who was previously under treatment with an alternate S1P1 modulator or agonist, and/or wherein said patient is currently undergoing discontinuation or cessation of treatment with an alternate S1P modulator or agonist.

In some embodiments said discontinuation or cessation of treatment is due to a bradycardia and/or lymphopenia event.

In some embodiments the medicament is in the form of a topical formulation selected from, for example, a solid, a patch, a powder, a liquid, a semisolid, an ointment, a gel, a spray, an aerosol, an inhaler and a lotion.

In some embodiments the medicament is an oral or injectable or systemic formulation, selected from, for example, a pill, a tablet, a capsule, a solution and a syrup.

In some embodiments the disease or disorder is an inflammation mediated disorder or immune mediated disorder, selected from, for example, the group consisting of psoriasis, eczema, vitiligo, prurigo nodularis, acne, rosacea, alopecia, rheumatoid arthritis, osteoarthritis, psoriatic arthritis, gout, stroke, haemorrhoid/piles, lung injury, liver injury, acute kidney injury, asthma, chronic obstructive pulmonary disease (COPD), uveitis, macular degeneration, glaucoma, otitis, allergy, sepsis, influenza, rhinitis, itch and pruritis.

In some embodiments the disease or disorder is pruritis, and preferably the medicament is an oral or topical formulation of a compound of Formula (I) as described herein.

In some embodiments the disease or disorder is acne and preferably the medicament is a topical formulation of a compound of Formula (I) as described herein.

In some embodiments the disease or disorder is rosacea and preferably the medicament is a topical formulation of a compound of Formula (I) as described herein.

In some embodiments the disease or disorder is a vascular mediated disorder, selected from the group consisting of, for example, aneurism, stroke, retinopathy, nephropathy, sepsis, kidney or liver injury and dilated vessel.

In some embodiments the disease or disorder is an autoimmune disorder, selected from, for example, the group consisting of multiple sclerosis and psoriasis.

In some embodiments, the disease or disorder is a vascular or central nervous system (CNS) disorder, selected from, for example, the group consisting of Parkinson's disease, Alzheimer disease, Motor neuron disease, Huntington disease, multiple sclerosis and neuropathy.

In some embodiments the subject is susceptible to heart failure, arrhythmias, high grade atrio-ventricular blocks, sick sinus syndrome, has a history of Syncopal episodes or a combination thereof.

In some embodiments the subject is undergoing beta blocker or anti-arrhythmic treatment by receiving anti-arrthymic drugs.

In some embodiments the subject has undergone an interruption or treatment break from another S1P receptor modulator/agonist. Said treatment break may be greater than 4, 6, 8, 10, 12, or 14 days.

In some embodiments, the medicament is administered topically, orally, transdermally, parenterally, intranasally, ocularly or rectally.

In some embodiments, the medicament is a slow release formulation, administered topically, by implantation or injection, or via a medical device.

In some embodiments the medicament is applied topically.

In some embodiments the medicament comprises the S1P receptor modulator in an amount between 0.01% and 30% by weight.

In one particular embodiment the medicament comprises the S1P receptor modulator in an amount of about 3% by weight.

In some embodiments the medicament is applied to up to 300 cm² of body surface area per 1 g of medicament formulation of various strength (i.e. 0.1% to 30% ww of active S1P receptor modulator), wherein the standard daily therapeutic dosage of S1P receptor modulator is ≤3 g. In one particular embodiment the standard daily therapeutic dosage of S1P receptor modulator is ≤1.5 g. In an alternative embodiment, the medicament is applied to up to 1000 cm² of body surface area per 1 g of medicament formulation of various strength (i.e. 0.1% to 30% ww of active S1P receptor modulator).

In some embodiments the medicament treats pain, selected from the group consisting of joint pain, arthritis pain, gout pain, back pain, muscle pain, neuropathic pain, neurologic pain, migraine, cancer pain, sports injury pain and wound pain.

In some embodiments the medicament comprises the S1P receptor modulator as a composition with one or more other pharmaceutically active compounds selected from, but not limited to, immune suppressant/modulators agents, pain modulators, pruritus modulators, neuromodulators, anti-inflammatory agents, antipathogens, antibacterial agents, antiviral agents and antifungal agents.

All prior art S1P1 modulators, tested in clinical trials or approved as a drug, induce bradycardia in humans after the initial dose (Bigaud M. et all, Biochimica et Biophysica Acta 2014, 1841, 745-758 and references therein; Pierre Eric J et al, Expert Opinion on Drug Metabolism & Toxicology, 2016, VOL. 12, NO. 8, 879-895; Pierre Eric J et al, Int. J. Mol. Sci. 2017, 18, 2636). The medicaments according to the present disclosure advantageously do not substantially affect heart rate after singular or multiple dosing to humans, and may therefore exhibit advantages in respect of treatment of certain indications.

Accordingly, in embodiments of the present disclosure, there is provided a method of treating or preventing a disease or disorder, comprising administering to a subject in need thereof a medicament, said medicament comprising a therapeutically effective amount of an SW receptor modulator, wherein the administration of the medicament does not cause a substantial decrease in heart rate. Preferably, the administration of the medicament does not cause bradycardia.

In a further embodiment of the present disclosure, there is provided a method of treating or preventing a disease or disorder, comprising administering to a subject in need thereof a medicament, said medicament comprising a therapeutically effective amount of an S1P receptor modulator, wherein the level of lymphopenia is between 25% and 70%. In an alternative embodiment, the level of lymphocytes is not altered.

According to embodiments of the present disclosure, a level of lymphopenia equivalent to, for example 25%, refers to a reduction in peripheral blood lymphocytes of 25% from the baseline value before the commencement of the initial daily dosage.

Severe lymphopenia is a significant side effect of S1P receptor modulator therapy and leads to a weakened immune system and other unwanted adverse effects (Pierre-Eric Juifa P-E. et al, Expert opinion on drug metabolism & toxicology, 2016, 12, (8), 879-895), (Johnson T A, Clinical Immunology (2010) 137, 15-20). Since S1P1 modulators may have utility in other non-autoimmune disorders, such as cardiovascular and neuronal diseases, due to its direct effect on endothelial and neuronal cells, there is a need for S1P modulators that do not cause lymphopenia, as it is an undesirable side effect that has no relevance to treatment outcomes in these indications. The methods according to the present disclosure result in no or reduced levels of lymphopenia and can be used safely in immune mediated and/or cardiovascular and/or neuronal and/or pruritus and/or pain indications where the preferred daily oral dose for treatment is ≤6 mg.

In any of the herein disclosed embodiments the compound of formula (I) may be administered in combination with other therapeutically active compounds, such as small molecules, biologicals, antivirals, antibacterial, pain modulators, pruritus modulators, anticancer drugs or anti-inflammatory agents.

Representative examples of the compound of formula (I) used in each of the above mentioned aspects and embodiments of the invention include, for example:

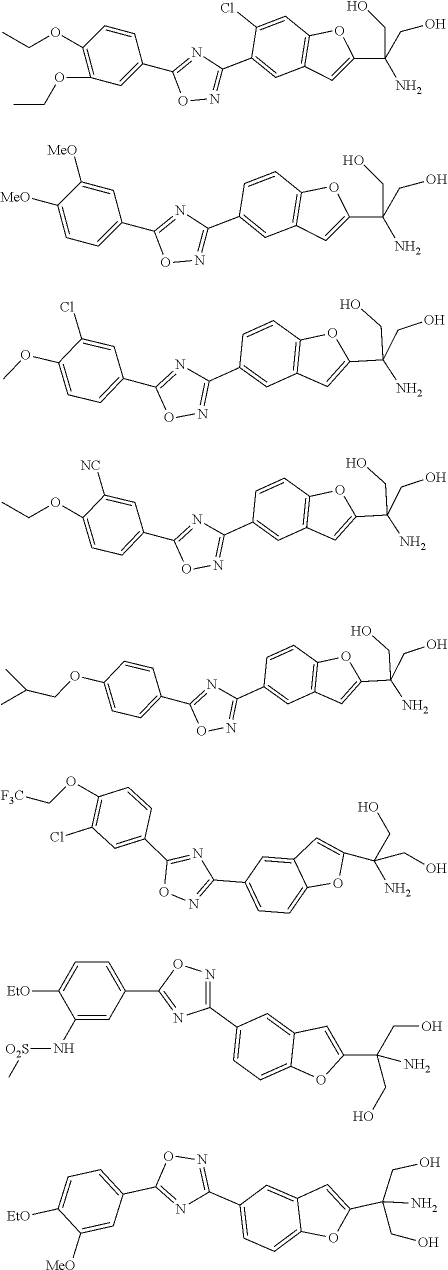

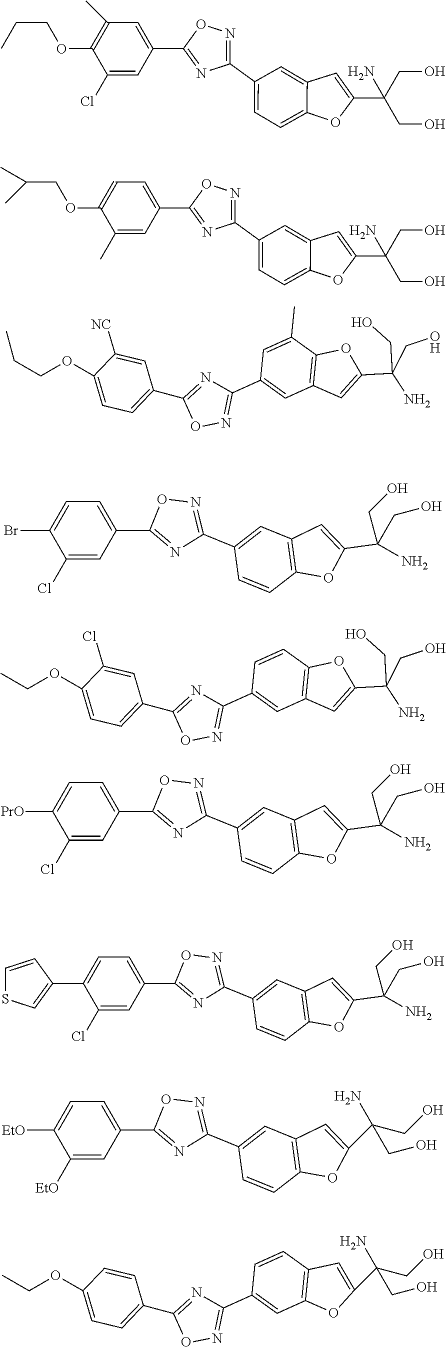

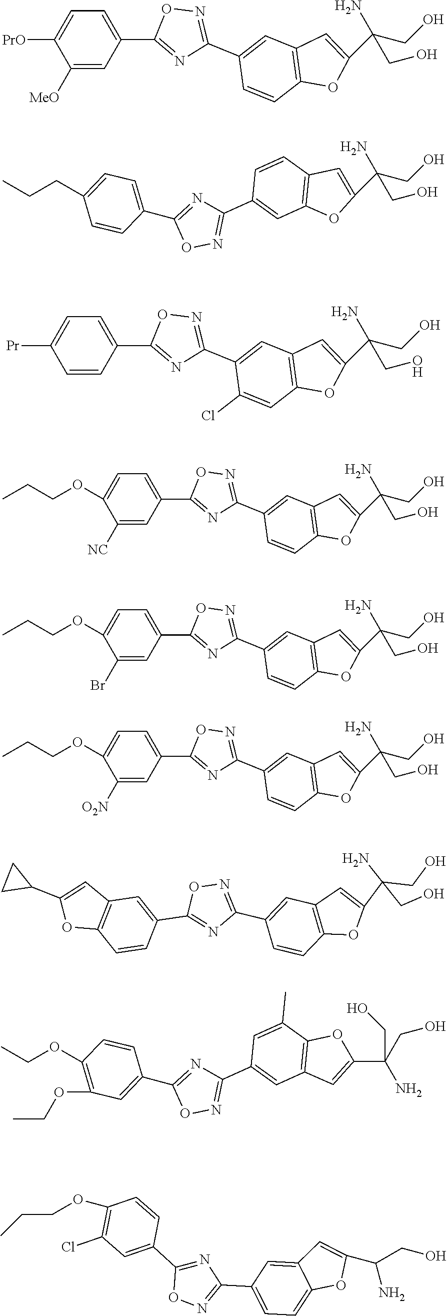

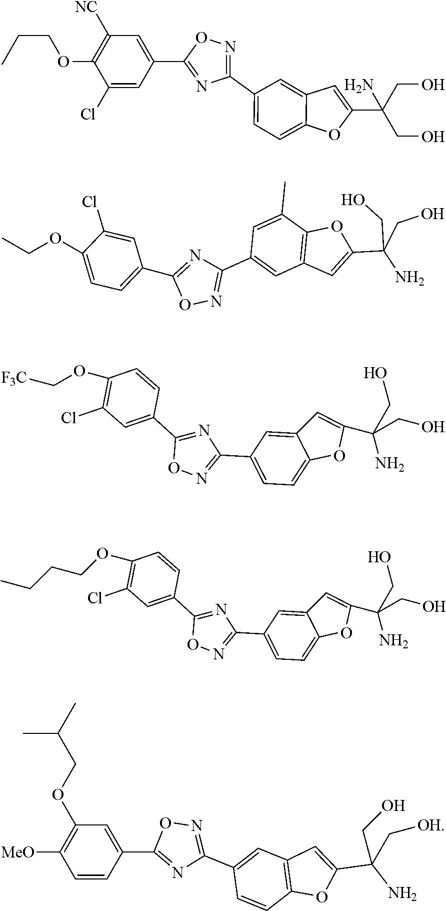 [SMILES: CCCOc1c(Cl)cc(-c2nc(-c3ccc4oc(C(N)(CO)CO)cc4c3)no2)cc1C#N ; CCOc1ccc(-c2nc(-c3cc(C)c4oc(C(N)(CO)CO)cc4c3)no2)cc1Cl ; NC(CO)(CO)c1cc2cc(-c3noc(-c4ccc(OCC(F)(F)F)c(Cl)c4)n3)ccc2o1 ; CCCCOc1ccc(-c2nc(-c3ccc4oc(C(N)(CO)CO)cc4c3)no2)cc1Cl ; COc1ccc(-c2nc(-c3ccc4oc(C(N)(CO)CO)cc4c3)no2)cc1OCC(C)C]

A preferred example of the compound of formula (I) is:

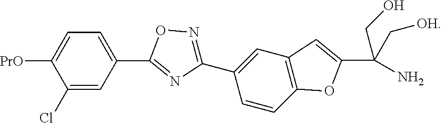

The S1P receptor modulator, for example the compound of formula 0), may be in the form of salts. The salts may be pharmaceutically acceptable. Suitable pharmaceutically acceptable salts will be apparent to those skilled in the art and include those described in J Pharm Sci, 1977, 66, 1-19, such as acid addition salts formed with inorganic acids for example hydrochloric, hydrobromic, sulfuric, nitric, boric or phosphoric acid; and organic acids for example succinic, maleic, acetic, fumaric, citric, tartaric, benzoic, p-toluenesulfonic, methanesulfonic or naphthalenesulfonic acid. Certain S1P receptor modulators, for example the compounds of formula (I), may form acid addition salts with one or more equivalents of the acid. The present disclosure includes within its scope all possible stoichiometric and non-stoichiometric forms and free base forms.

The S1P receptor modulator, for example the compounds of formula (I), may be prepared in crystalline or non-crystalline form, and, if crystalline, may optionally be hydrated or solvated. This disclosure includes within its scope stoichiometric hydrates or solvates as well as compounds containing variable amounts of water and/or solvent and all salts, solvates, hydrates, complexes, polymorphs, prodrugs, radiolabeled derivatives, stereoisomers and optical isomers of the S1P receptor modulator, for example the compounds of formula (I).

The presently disclosed medicaments may contain the S1P receptor modulators, for example, the compound of formula (I) as the only active ingredient and optionally one or more inactive ingredients. Alternatively, the medicaments may include compositions, wherein the compound of formula (I) is combined with one or more other active ingredients, and, optionally, inactive ingredients.

In some embodiments. the medicament may comprise a variety of delivery vehicles such as pharmaceutical excipients, including stabilizing agents, carriers or encapsulation formulations for the systemic (i.e. oral, injectable, device) or local or targeted use (i.e. topical, ear, eye, nasal, oral, parenteral, rectal). The compositions may provide a favourable combination effect between, for example at least one compound selected from one or more of the group consisting of but not limited to steroids, opioids and non-steroidal anti-inflammatory drugs, cannabinoids such as cannabidiol (CBD) and the delivery vehicles. The combined effect may improve treatment and/or prevention and/or immunotherapy in comparison to the S1P receptor modulator, for example the compounds of formula (I), alone.

Other active and non-active ingredients or excipients include, but are not limited to ointments, gels, hydrogel, solution, drops, topical patches, transdermal patches, topical liquid preparations, sprays, aerosols, lotion, foam, controlled degrading polymers, patches, tablets, capsules, oral liquid preparations, powders, granules, lozenges, controlled release particles including microparticles, liposomes, nano-emulsions, polymers, microsponges or fullerenes, injectable or infusible solutions or suspensions or suppositories and others.

In another aspect of the present disclosure there is provided a method of treating or preventing a disease or disorder, comprising administering to a subject in need thereof a medicament, said medicament comprising a therapeutically effective amount of an S1P receptor modulator, wherein the medicament is administered to a patient who was previously under treatment with an alternate S1P1 modulator or agonist, and/or wherein said patient is currently undergoing discontinuation or cessation of treatment with said alternate SW modulator or agonist.

In another aspect of the present disclosure there is provided a therapeutically effective amount of an S1P receptor modulator for use in treating or preventing a disease or disorder, wherein the medicament is administered to a patient who was previously under treatment with an alternate S1P1 modulator or agonist, and/or wherein said patient is currently undergoing discontinuation or cessation of treatment with said alternate S1P modulator or agonist.

In another aspect of the present disclosure there is provided use of a therapeutically effective amount of an S1P receptor modulator in the preparation of a medicament for treating or preventing a disease or disorder, wherein the medicament is for a patient who was previously under treatment with an alternate S1P1 modulator or agonist, and/or wherein said patient is currently undergoing discontinuation or cessation of treatment with said alternate SW modulator or agonist.

As described previously, other S1P modulator therapy may result in the emergence of additional side effects including cardiovascular abnormalities and lymphopenia. The emergence of these side effects may necessitate a treatment break of the S1P modulator or agonist, during which the control of the disease or disorder may worsen. Such patients may, during this period of cessation of treatment, instead be treated by the methods according to the present disclosure due to the reduced observation or absence of any side effects.

In some embodiments of this aspect, the discontinuation or cessation of treatment is due to a bradycardia or lymphopenia event. In some embodiments, the discontinuation or cessation of treatment is due to the emergence of both bradycardia and lymphopenia events.

Accordingly there is provided a method of preventing or ameliorating the risk of bradycardia in a subject receiving a medicament comprising a therapeutically effective amount of an S1P receptor modulator, wherein the medicament is administered to a subject who was previously under treatment with an alternate S1P1 modulator or agonist, and/or wherein said patient is currently undergoing discontinuation or cessation of treatment with said alternate S1P modulator or agonist.

There is also provided a method of preventing or reducing the level of lymphopenia in a subject receiving a medicament comprising a therapeutically effective amount of an S1P receptor modulator, wherein the medicament is administered to a subject who was previously under treatment with an alternate S1P1 modulator or agonist, and/or wherein said patient is currently undergoing discontinuation or cessation of treatment with said alternate SW modulator or agonist.

Preferably in each of the abovementioned embodiments of the invention, the medicament decreases the heart rate of the subject by about 5 beats/min or less daily, or about 4 beats/min or less daily, or about 3 beats/min or less daily, or about 2 beats/min or less daily, and more preferably the S1P receptor modulator is administered at an initial daily dosage which is substantially the same as the standard daily therapeutic dosage.

In any of the herein disclosed methods the medicament may be topical and in the form of a liquid formulation, such as and not limited to lotion and solution, semisolid formulations such as and not limited to ointment, gel, foam or cream, sprays and aerosols, or solid formulation such as and not limited to topical patches. The topical delivery systems may also include aerosol foams, liposomes, nano-emulsions, polymers, microsponges or fullerenes (Pharma Innovation, 2012, 1(9), 18-31). A topical composition may contain skin penetration enhancers. Examples of skin penetration enhancers include, but are not limited to short chain alcohols, such as dimethylsulphoxide, dimethyl isosorbides, stearic acid, ethanol, propylene glycol and isopropanol; long chain alcohols such as decanol, hexanol, lauryl alcohol, myristyl alcohol, octanol, octyl dodecanol, cetyl alcohol, stearyl alcohol, oleyl alcohol; cyclic amides, such as azone; esters, such as ethyl acetate, octyl salicylate, padimate O, ethyl oleate, glyceryl stearates, glyceryl monoleate, glyceryl monocaprate, glyceryl tricaprylate, isopropyl myristate, isopropyl palmitate, propylene glycol monolaurate, or propylene glycol monocaprylate; fatty acids such as lauric, linoleic, myristic, oleic, palmitic, stearic or isostearic acids; glycols such as dipropylene, propylene, 1,2-butylene or 1,3-butylene glycols; pyrrolidones, such as N-methyl-2-pyrrolidone, or 2-pyrrolidone; sulphoxides, such as decylmethyl sulphoxide or dimethyl sulphoxide; anionic surfactants such as sodium lauryl sulphate, cationic surfactants such as alkyl dimethylbenzyl ammonium halides, alkyl trimethyl ammonium halides, alkyl pyridinium halides, non-ionic surfactants, such as Brij 36T or Tween 80; monoterpenes, such as eugenol, d-limonene, menthol, menthone; sesquiterpenes, such as farnesol or neridol.

In some embodiments of the present disclosure, the inflammation mediated disorder or immune mediated disorder may be selected from the group consisting of and not limited to psoriasis, eczema, vitiligo, prurigo nodularis, alopecia, rheumatoid arthritis, osteoarthritis, gout, haemorrhoid/piles, asthma, chronic obstructive pulmonary disease (COPD), uveitis, retinopathy, nephropathy, macular degeneration, glaucoma, otitis, allergy, sepsis, influenza, rhinitis and pruritus. In one preferred embodiment of the present disclosure, the disease or disorder is pruritus.

In some embodiments of the present disclosure, the methods may be used in transplantation purposes, such as, but not limited to, cornea, kidney and liver transplants.

In some embodiments of the present disclosure, the vascular mediated disorder may be selected from the group consisting of and not limited to aneurism, vessel inflammation, stroke, heart attack, ischemic injury, Peripheral artery disease, lung injury, liver injury, kidney injury, retinopathy, nephropathy, hemorrhoids, blood vessel abnormalities and inflammations, vasculopathy, aneurism, chronic wounds or leg ulcers.

In some embodiments of the present disclosure, the vascular or central nervous system (CNS) disorder may be selected from the group consisting of and not limited to Multiple Sclerosis, Parkinson disease, Alzheimer disease, Huntington disease, Motor Neuron disease, epilepsy, tension headache, anxiety, ALS, neuromuscular disease, neuropathy.

The present disclosure also provides a method of treating or preventing pain comprising administering to a subject in need thereof a medicament, said medicament comprising a therapeutically effective amount of an S1P receptor modulator according to any one of the herein disclosed embodiments.

The present disclosure also provides a therapeutically effective amount of an S1P receptor modulator according to any one of the herein disclosed embodiments for use in a method of treating or preventing pain.

The present disclosure also provides use of a therapeutically effective amount of an S1P receptor modulator according to any one of the herein disclosed embodiments in the preparation of a medicament for treating or preventing pain.

In some embodiments, the disease or disorder is a condition, selected from but not limited to the group consisting of: susceptible heart failure, arrhythmias, high grade atrio-ventricular blocks, sick sinus syndrome, history of Syncopal episodes or a combination thereof.

In an embodiment, the subject is undergoing beta blocker or anti-arrhythmic treatment by receiving anti-arrhythmic drugs.

In an embodiment, the patient has undergone an interruption or treatment break from another S1P receptor modulator/agonist. The treatment break may be greater than 4, 6, 8, 10, 12, or 14 days.

As prior art S1P receptor modulator/agonists have been known to result in bradycardia or lymphopenia events, patients may be required to take treatment breaks to reduce the bradycardia or lymphopenia events before treatment can continue. Advantageously, as the medicaments of the present disclosure may not result in bradycardia and/or significant lymphopenia events, these patients may then continue to be treated by the present medicaments.

In any one of the herein disclosed methods the medicament may be administered by any acceptable mode of administration, for example topically, orally, intravenous, parenterally, intranasally, ocularly or rectally.

In some embodiments of the herein disclosed methods may be used to treat gastrointestinal problems such as, but not limited to, gut inflammations, vessel abnormalities, wounds, ulcers, hemorrhoids, pruritus ani, ulcerative colitis and Crohn's disease.

In any of the herein disclosed methods the medicament may be a slow release formulation, administered topically or by implantation or injection or via a medical device. The slow release medicaments may have a desirable therapeutic level of the the S1P receptor modulator at a systemic or local level. The slow release medicament may also be applied at the in-situ or periphery of an affected part, for example, inflammation, ischemic injury, cancer, tumor, atherosclerotic lesion.

When applied locally, the slow release medicament may be applied without an associated increase in systemic exposure. The process may enhance the overall therapeutic window which otherwise may not be possible via systemic treatment. For example, a skin lesion of psoriasis or atopic dermatitis (eczema) may receive the required exposure to a S1P receptor modulator by direct administration to the lesion of a medicament according to the present disclosure, while a systemic treatment may not achieve adequate therapeutic exposure of a S1P receptor modulator.

The methods according to the present disclosure may be used to treat the indications via oral, systemic or local to, for example, skin, eye, ear, nose, lungs, mouth, rectal and anal or the gastrointestinal organs via a slow releasing formulation, The treatment of hypoxia, for example at the remote part of cancer, by local administration of an effective amount of the presently disclosed medicament to a subject in need. Transplant rejection is often accompanied by inflammation (Lutz et al, J Inflamm (Lond), 2010, 7, 27; Liang J et al, Cornea, 2014, 33 (4), 398). S1P receptor modulation is involved in an immune tolerance and vasculature correction and a local administration and optimal exposure may be a promising approach for successful transplants with or without other immune modulators. Accordingly, there is provided a method of treating transplant rejection by local administration, comprising administering to a subject in need thereof a medicament, said medicament comprising a therapeutically effective amount of an S1P receptor modulator. S1P receptor modulation can mount an effective and appropriate response which spans from immune action against infection (Pinschewer D. D. et al, Neurology, 2011, 76 (Suppl 3): S15-S19) or cancer (Marcus A et al, Blood, 2011, 118(4), 975) to the immune tolerance (Liu G. et al, J Immunol, 2014, 192; Yoshida Y. et al, Biol Pharm Bull, 2011, 34(6), 933) and transplant success.

The topical formulations, tablets and capsules according to the present disclosure for oral administration may be in unit dose form, and may contain conventional excipients, such as binding agents (e.g. pregelatinised maize starch, polyvinylpyrrolidone, hydroxyethyl or hydroxypropyl methylcellulose); fillers (e.g. lactose, microcrystalline cellulose or calcium hydrogen phosphate); tableting lubricants (e.g. magnesium stearate, talc or silica); disintegrants (e.g. potato starch or sodium starch glycolate); and acceptable wetting agents (e.g. sodium lauryl sulphate). The tablets may be coaled according to methods well known in normal pharmaceutical practice. The tablets may be slow releasing and release in specific organs, such as stomach or intestines, to deliver the S1P receptor modulator, for example the compounds of formula (I).

The topical and oral liquid formulations of the present disclosure may be in the form of aqueous or oily suspension; solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for topical and oral administration may be suitably formulated to give controlled release of S1P receptor modulators.

For parenteral administration, fluid unit dosage forms may be prepared utilizing a compounds of formula (I) or pharmaceutically acceptable salts thereof and a sterile vehicle. Formulations for injection may be presented in unit dosage form e.g. in ampoules or in multi-dose, utilizing a compounds of formula (I), or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g. sterile pyrogen-free water, before use. In preparing solutions, the compound of formula (I) can be dissolved for injection and filter sterilized before filling into a suitable vial or ampoule and sealing. Advantageously, adjuvants such as a local anaesthetic, preservatives and buffering agents may be dissolved in the vehicle. A surfactant or wetting agent may be included in the composition to facilitate uniform distribution of the compound.

Lotions may be formulated with an aqueous or oily base and may also contain one or more emulsifying agents, stabilizing agents, dispersing agents, suspending agents, thickening agents, or coloring agents. Drops may be formulated with an aqueous or non-aqueous base also comprising one or more dispersing agents, stabilizing agents, solubilizing agents or suspending agents. They may also contain a preservative.

In any of the herein disclosed methods the medicament may also be formulated in rectal compositions such as suppositories or retention enemas, e.g. containing conventional suppository bases such as cocoa butter or other glycerides. As depot preparations and such long acting formulations may be administered by implantation (for example subcutaneously or intramuscularly, in-situ, at the periphery of inflammatory and/or injury site) or by intramuscular injection. May be formulated with suitable polymeric or hydrophobic materials (for example as an emulsion in an acceptable oil) or ion exchange resins, or as sparingly soluble derivatives, for example, as a sparingly soluble salt.

For a formulation as controlled release particles the required amount of the compounds of formula (I) may be treated with a polymer, specifically biodegradable polymers which degrade in vivo, either enzymatically or non-enzymatically or both, to produce biocompatible, toxicologically safe by-products which are further eliminated by normal metabolic pathways. The choice of such biodegradable polymers includes, but is not limited to, poly lactic-coglycolic acid (PLGA) polyanhydrides, PLAGA, polycaprolactone (PCL), complex sugars (hyaluronan, hitosan) and inorganics (hydroxyapatite). For better delivery formulations that incorporate a compound of formula (I) with various types of block copolymers of polyesters with poly ethylene glycol (PEG). PLGA/PEG block copolymers as diblock (PLGA-PEG) or triblock molecules with both ABA (PLGA-PEG-PLGA) and BAB (PEG-PLGA-PEG). These drug delivery devices may avoid the inconvenient surgical insertion of large implants and the injectable biodegradable and biocompatible PLGA particles (microspheres, microcapsules, nanocapsules, nanospheres) may be employed for controlled-release dosage forms. The active ingredients may be released from polymeric devices either by diffusion through the polymer barrier, or by erosion of the polymer material, or by a combination of both diffusion and erosion mechanisms.

For intranasal administration, the compounds of formula (I) or pharmaceutically acceptable salts thereof, as alone or compositions with other active ingredients may be formulated as solutions for administration via a suitable metered or unitary dose device or alternatively as a powder mix with a suitable carrier for administration using a suitable delivery device. Thus, compounds of formula (I) or pharmaceutically acceptable salts thereof and/or combinations may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof, alone or as compositions with other active ingredients, may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilized components. Compounds of formula (I) or pharmaceutically acceptable salts thereof may be used as alone or in combination preparations with other therapeutically active compounds, such as and not limited to cyclosporin A, methotrexate, steroids, corticosteroids, non-steroidal anti-inflammatory drugs, inflammatory cytokine inhibitors, kinase inhibitor (e.g., JAK Kinase), immunomodulators including biologicals, antivirals, including but not limited to aciclovir, 5-fluorouracil, galancyclovir, valancyclovir, vidaramine or zidovudine, and broad spectrum antiviral agents (Front Microbiol, 2015; 6: 517), antibiotics, including but not limited to amoxicillin, ceftaroline, colistin, dyptomycin, ertapenem, fosfomycin, penicillin, rapamycin or tigecyline; or antifungals, including but not limited to amphotericin, liposomal amphotericin B, fluconazole, flucytosine, micafungin, posaconasole and viriconazole.

The compounds of formula (I) or pharmaceutically acceptable salts thereof may be used as alone or in combination preparations according to the present disclosure may be used for the treatment of other diseases or disorders including but not limited. to atherosclerotic lesions, tumors, kidney (nephropathy), prostate (prostatitis), urinary tract (inflammations), pancreases (pancreatitis), colon (colitis), liver (hepatic diseases, deep tissue (neuropathy, inflammations, degenerations), ulcers, wounds, ischemic injury, bone regeneration, muscle regeneration, epithelial ulcer treatment, wound healing, therapeutic angiogenesis and gangrene. The formulation may be administered at, or at the periphery of, an affected area.

The medicaments according to the present disclosure may contain from about 0.05% to about 10% by weight, or from about 0.5% to about 10% by weight, preferably from about 1.0 to about 4.5% by weight or preferably from about 1.5 to about 4.5% by weight, of the S1P receptor modulator. The systemic exposure with 2 g daily topical 3% ww formulation treatment for 3 weeks of compound of formula I gave average systemic exposure of ˜2.5 ng/mL and the oral dose of 8 mg daily for one week gave average systemic exposure of ˜53 ng/mL without event of bradycardia and significant lymphopenia. The dose of the S1P receptor modulator used in the treatment of the aforementioned disorders will vary in the usual way with the seriousness of the disorders, the weight of the sufferer, and other similar factors. In preferred embodiments, the medicament is applied to 100 cm² of body surface area per 1 g of medicament, up to 1000 cm² of body surface area per 1 g of medicament. The medicament can be applied such that the standard daily therapeutic dosage of active compound is ≤3 g. The topical dose level and strength of dose formulation with the compound of formula (I) may be extended to higher levels without risk of bradycardia and lymphopenia. For example, dose levels of medicaments according to the methods of the present disclosure may include up to:
1. 10 g daily dose of up to 30% ww strength formulation with 1 g up to 300 cm² surface area
2. 10 g daily dose of up to 30% ww strength formulation with 1 g up to 1000 cm2 surface area
3. 100 g daily dose of up to 3% ww strength formulation with 1 g up to 300 cm² surface area
4. 100 g daily dose of up to 3% ww strength formulation with 1 g up to 1000 cm2 surface area
5. 300 g daily dose of up to 1% ww strength formulation with 1 g up to 300 cm² surface area.
6. 300 g daily dose of up to 1% ww strength formulation with 1 g up to 1000 cm² surface area

According to methods of the present disclosure, the disease or disorder may be pain selected from the group consisting of but not limited to neuralgia, migraine, nociceptive pain, neuropathic pain, inflammatory pain, wound pain, tension headache, herpetic neuralgia, muscle pain, joint pain, back pain, wound pain, sports injury pain, and so forth.

As a slow release formulation the medicament may be formulated to daily release active compound of up to 70 mg.

### BRIEF DESCRIPTION OF THE DRAWINGS

FIG. 1 illustrates various S1P receptor modulator pathways.

FIG. 2 illustrates the effect of a compound of Formula I on markers of inflammation and pruritis.

FIG. 3 illustrates the effect of a compound of Formula I on pruritus scores.

FIG. 4 illustrates the effect of a compound of Formula I on heart rate of human subjects over 72 hours, at a 12 mg dose.

FIG. 5 illustrates the anti-mould activity of the compound of formula I solution.4

FIG. 6 illustrates the effect of compound of formula I on the secretion of cytokines TNFα and IL6.

FIG. 7 confirms that the compound of formula (I) did not inhibit or impair wound healing.

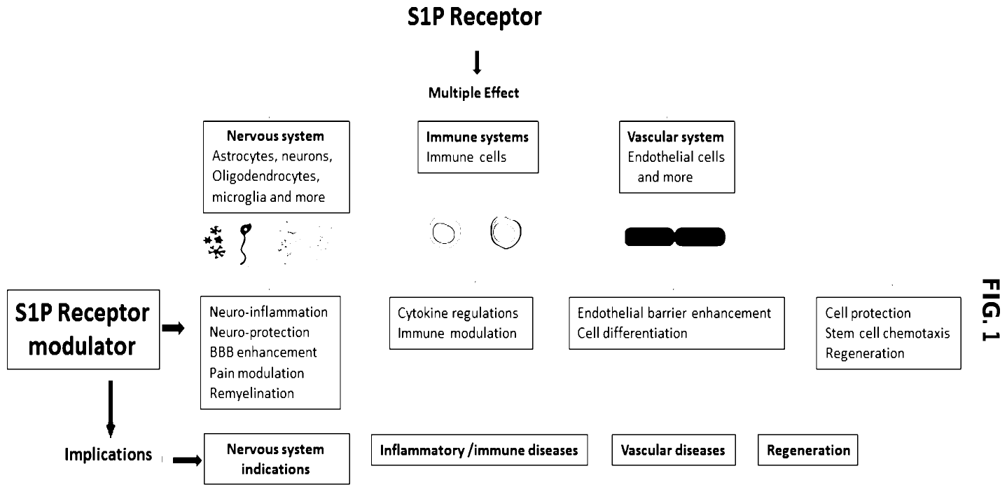

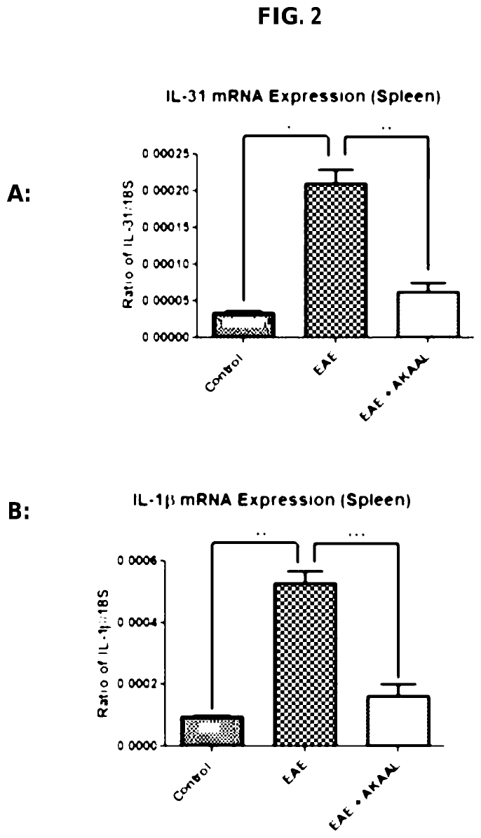

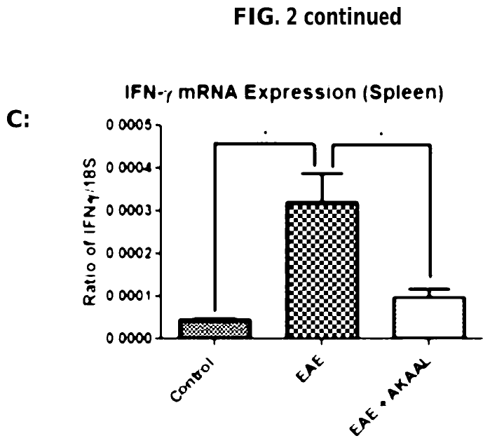

### DETAILED DESCRIPTION OF EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure.

Although any methods and compositions similar or equivalent to those described herein can also be used in the practice or testing of the present disclosure, the preferred methods and compositions are now described.

It must also be noted that, as used in the specification and the appended claims, the singular forms ‘a’, ‘an’ and ‘the’ include plural referents unless otherwise specified. Thus, for example, reference to ‘S1P receptor modulator’ may include more than one S1P receptor modulator, and the like.

Throughout this specification, use of the terms ‘comprises’ or ‘comprising’ or grammatical variations thereon shall be taken to specify the presence of stated features, integers, steps or components but does not preclude the presence or addition of one or more other features, integers, steps, components or groups thereof not specifically mentioned.

Unless specifically stated or obvious from context, as used herein, the term ‘about’ is understood as within a range of normal tolerance in the art, for example within two standard deviations of the mean. ‘About’ can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein in the specification and the claim can be modified by the term ‘about’.

Any methods provided herein can be combined with one or more of any of the other methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to exemplary embodiments of the disclosure. It is understood that the detailed examples and embodiments described herein are given by way of example for illustrative purposes only, and are in no way considered to be limiting to the disclosure.

### EXAMPLES

The compound of formula (I) used in the below examples was:

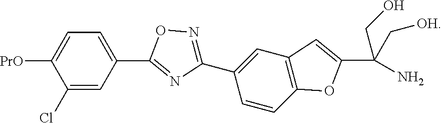

### Example 1: Activity at Spingosine-1-Phosphate (S1P) Receptor

Compounds of formula (I) showed S1P receptor activity, especially type 1 receptor agonistic activity. The S1P′ assay system was GTPgama-S³⁵ binding in membranes from CHO K1 cells, expressing S1P′ human receptor. The compounds were tested and generated a concentration-effect (dose response) curves at these receptors. The analysis provided efficacy (Eₘₐₓ) and potency (EC₅₀) of selected compounds of Formula (I) relative to S1P and demonstrated an EC₅₀ of <2 nM at the S1P₁ receptor. The compounds of Formula I has low tendency to degrade the S1P₁ receptor. The compounds of formula (I) are selective against the S1P2, S1P3, S1P4 and S1P5 receptors.

Comparatively, the endogenous ligand S1P and drug FTY-720 has activity at S1P1 receptor in a GTPγS assay with EC50 nM of 1.2 nM and 2 nM respectively while the receptor degradation ability is 302 nM and 0.34 nM respectively (Lucas et al, Journal of Biomolecular Screening, 2013, 1-10 pp). The known S1P receptor modulators have low activity v/s degradation margin FTY720=0.17; BAF-312=2.95, Ponesimod <2 while the compound of formula (I) has a margin greater than 100 (Samuvel J et al, PLOS ONE, 2015, DOI:10.1371/journal.pone.0141781; Piali L et, JPET, 2011, 337, 547-556; Lucas S et al, Journal of Biomolecular Screening, 2014, Vol. 19(3) 407-416; Gatfield J et al, Immunomodulation, 28th ECTRIM, 10-13 Oct. 2012, Lyon, France).

### Example 2: Correlation with Lymphopenia and Systemic Exposure of Compound of Formula (I) in Humans and Mice

Treatment of a compound of formula (I) (dose of ˜0.17 mg/kg) in humans resulted in a systemic exposure of 18.8 ng/mL with no induction of significant lymphopenia. In comparison, treatment of a compound of formula I (dose of 0.3 mg/kg) in mice resulted in a systemic exposure of 6.36 ng/mL with a measured lymphopenia of 40% at 6 hours post dose. This is surprising and suggests that the link between lymphopenia and the systemic exposure of S1P receptor modulators such as the compound of formula (I) differs between mice and humans.

It is notable that the delivery of a compound of formula (I) had a dose proportional systemic exposure. The 8 mg daily dosing of compound of formula (I) for one week resulted in a measured lymphopenia of only up to 40%, which is a dosage 16 times higher than the equivalent FTY720 therapeutic dose in humans.

### Example 3: Effects of a Compound of Formula (I) In-Vivo on Heart Rate

Treatment of a compound of Formula I in humans resulted in a dose proportional increase in systemic exposure in humans with 0.5 mg=0.6 ng/mL; 2 mg=2.4 ng/mL; 6 mg=7.1 ng/mL; 12 mg=18.8 ng/mL; however, there was no dose proportional or dose relevant bradycardia event, a common side effect observed in prior art S1P1 modulators/agonists where the decrease of heart rate is sustained for several hours.

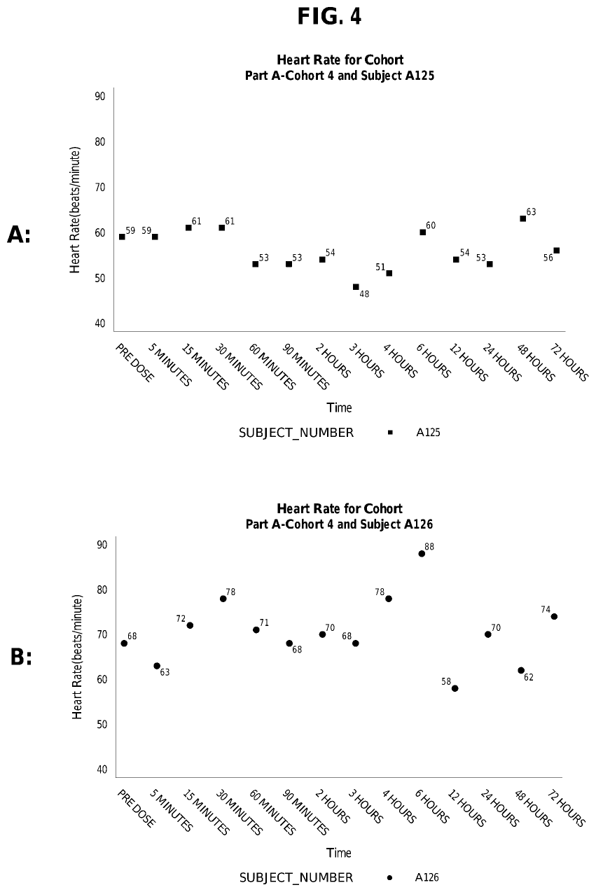

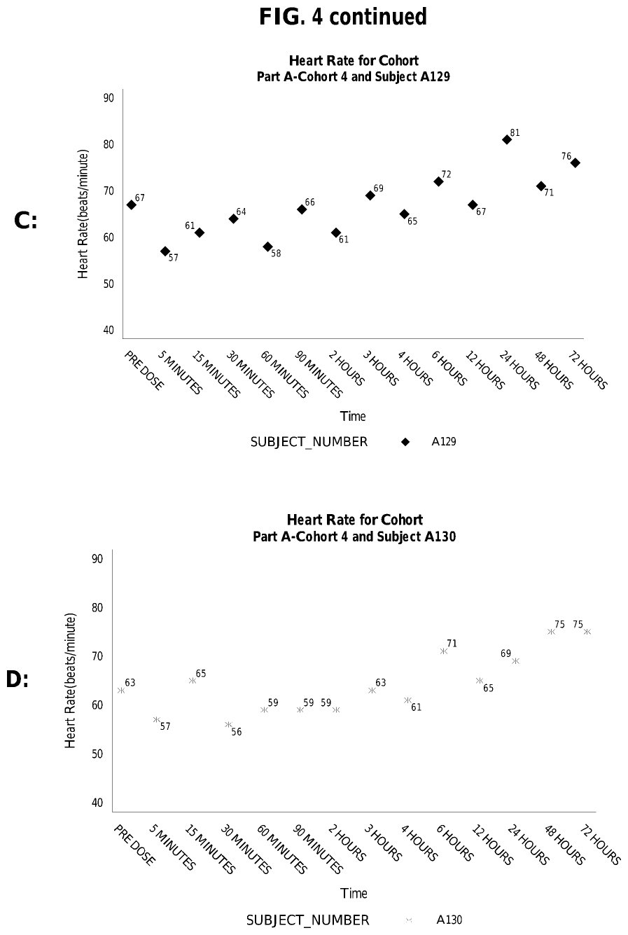

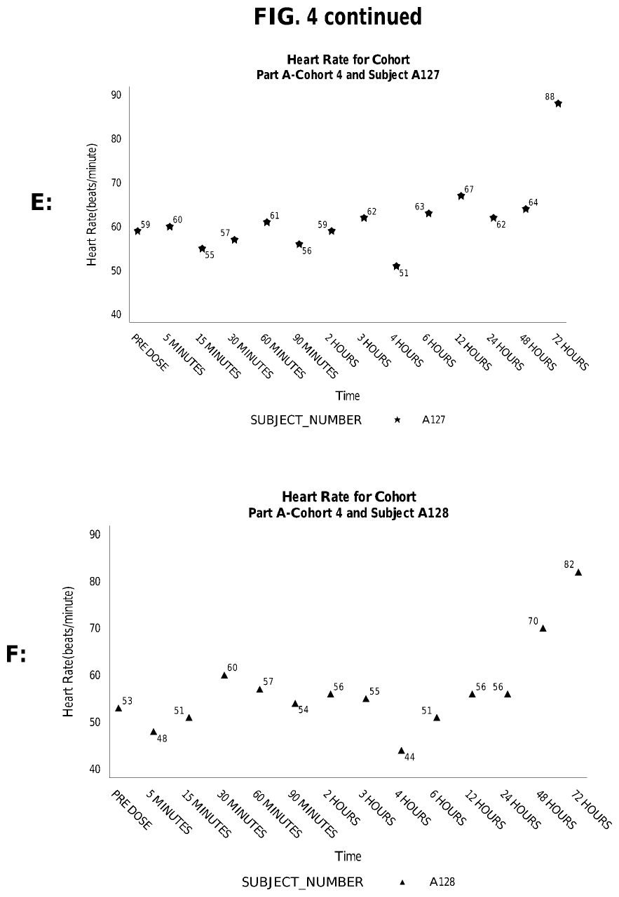

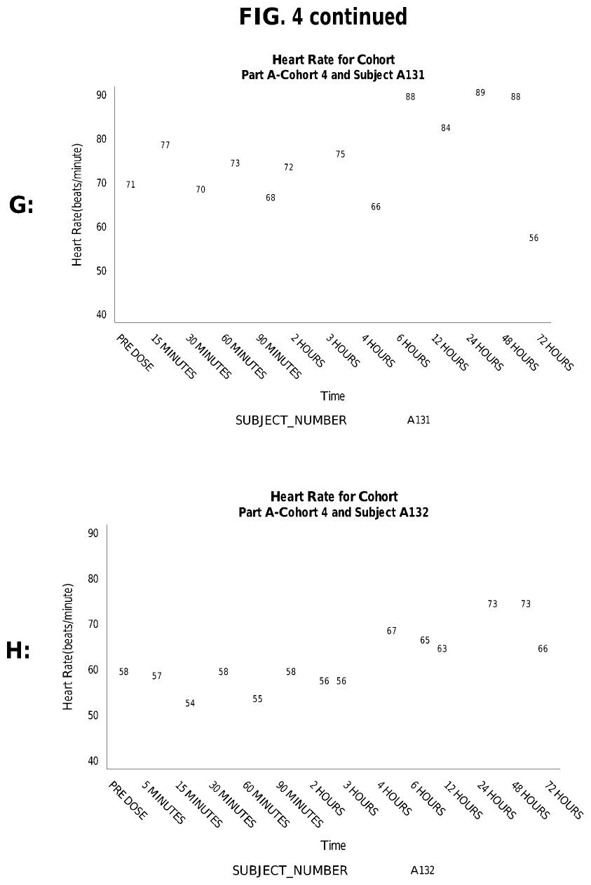

The observance of bradycardia with S1P1 selective agonists has been reported to be species specific. S1P1 receptor selective agonists do not induce bradycardia in rodents; however the S1P1 selective agonists tested in humans resulted in significant bradycardia in patients (Juif P-E. et al, Int. J. Mol. Sci. 2017, 18, 2636; doi:10.3390/ijms18122636; Pali L. et al, Pharmacol Res Perspect, 2017; e00370. wileyonlinelibrary.com/journal/prp2|1 of 12; Rey M et al, PLOS ONE, September 2013|Volume 8|Issue 9|e74285). Surprisingly the S1P1 receptor modulator of formula (I) did not induce bradycardia in humans when tested at different dose levels ranging from 0.5 mg dose to 12 mg dose (12 mg data shown in FIG. 4).

### Example 4: Efficacy of a Compound of Formula (I) in an Animal Model of Excision Wound

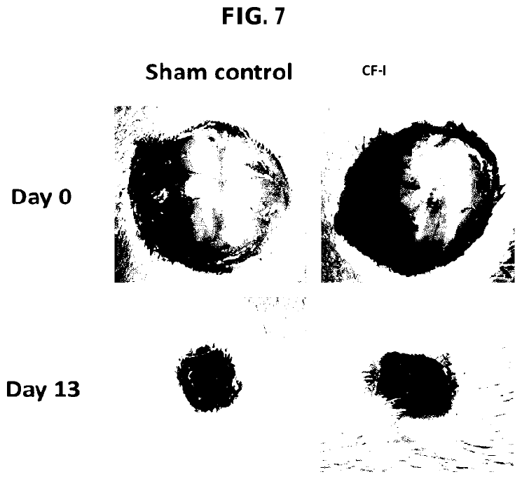

Two groups with six Wistar rats in each group were anaesthetized with a dose of 80 mg/kg of ketamine (i.p.) and the back of the animals were shaved. One excision wound was inflicted by cutting away (with a sterile scalpel) a 600-700 mm² section of the full thickness of the skin from a predetermined area. The wound was left undressed to the open environment. Group 1 rats were untreated and served as sham control. Group 2 animals were treated with a compound of Formula I in an ointment formulation (3% w/w). The ointment (0.20 g/animal wound) was applied topically twice daily. The compound of formula (I) did not inhibit or impair the wound healing (FIG. 7).

### Example 5: Efficacy of a Compound of Formula (I) In-Vitro for Inflammation

Treatment of a compound of formula (I) (1-5 μg/mL) to the cultured microglial (BV2 cells) and macrophages (Raw cells) was made 4 hrs prior to treatment with lipopolysaccharide (LPS) at 500 ng/mL. After 16-18 hr of drug treatment the cytokines (TNF_{α}, IL_{1β} and IL6) were measured in culture media by ELISA and expression levels of cyclooxygenase2 (Cox-2), inducible nitroxide synthase (iNOS) or beta actin were analysed in cell homogenates by western analysis. There was a significant reduction of proinflammatory cytokines (p<0.05) when treated with a compound of formula (I).

### Example 6: Efficacy of Compound of Formula (I) in In-Vivo for Pruritus and Inflammation

The efficacy of the compound of formula (I) was assessed in an animal model of experimental autoimmune encephalomyelitis (EAE), which is a widely-accepted model of demyelinating diseases such as MS. The cDNA was prepared from spleen obtained from Vehicle treated (Control), EAE induced (no treatment) and EAE induced (a compound of formula (I) treated) animals (n=5 each group) at the recovery (Day 27 from 1^{st} day of MBP immunization). The treatment with a compound of Formula I (3 mg/kg) was initiated on the 11^{th} day from MBP immunization. cDNA was synthesized using Bio-Rad cDNA synthesis kit. RT-PCR was performed for IL-31, IL1β and IFNγ. Primers were obtained from Qiagen and the samples were analysed on Bio-Rad CFX96Real-Time System. Administration of a compound of formula (I) significantly reduced markers of inflammation (Figure-2). Statistical Significance of the data is represented by p value. *denotes p≤0.05, **denotes p≤0.01.

In an atopic dermatitis and psoriasis disease models the compound of formula (I) downregulates the cytokines IL17, IL23, IL2.

### Example 7: Efficacy of Compound of a Formula (I) in an Animal Model of Formalin Induced Nociception (Pain)

Forty male Sprague Dawley rats were assigned to different treatment and control groups containing 8 animals/group. The groups were treated with different doses of a compound of Formula I and received either 0.03, 0.3 and 3.0 mg/kg dose, administered orally 30 min before formalin injection. Tramadol hydrochloride (30 mg/kg) was administered orally 30 min before formalin injection as a positive control. Deionized water was used as a negative control (Vehicle) and administered orally 30 min before formalin injection. Individual rats were then gently restrained and formalin (5% in v/v in saline, 50 μl, s.c.) was injected into the plantar surface of the left hind paw using a 27G needle. Post-formalin nociceptive behaviour, paw flinching was recorded for 60 min, in bins of 5 min. The intra plantar injection of formalin (50 μl, 5% v/v) induced marked flinching of the ipsilateral rat paw with typical biphasic response in the vehicle/distilled water treated animals. The compound of formula (I) at 0.03 mg/kg, 0.3 mg/kg and 3 mg/kg when administered orally 30 min before formalin treatment inhibited nociceptive behaviour dose. Inhibition of nociceptive behaviour at 0.3 mg/kg (33%, p<0.05) and 3.0 mg/kg (45%, p<0.01) were statistically significant compared to vehicle control.

### Example 8: Efficacy of Compound of a Formula (I) in an Animal Model of Paclitaxel Induced Neuropathic Pain

Forty-eight male Sprague Dawley Rats were assigned to different treatment and control groups containing 8 animals per group. All animals, except the Naive control were injected with Paclitaxel (2 mg/kg) intraperitoneally on four alternate days (D0, D2, D4 and D6). For prophylactic intervention, the compound of Formula I was dosed orally at dose of 0.3, 1 and 3 mg/kg/day when the treatment was initiated along with paclitaxel treatment and the dosage was continued until day 15 post first paclitaxel injection. For therapeutic intervention, the compound of Formula I was dosed orally at dose of 3 mg/kg/day when the treatment was initiated after establishment of neuropathic pain i.e. on day 16 post first paclitaxel injection and was continued for the next 7 days.

For behavioural testing each rat was allowed to acclimatize individually to a transparent enclosure on elevated wire mesh for at least 15 min. Behavioural testing was performed using an Electronic von Frey Aesthesiometer (IITC Life Science) to evaluate paw withdrawal threshold to mechanical stimulus. Paclitaxel injections in negative control (Paclitaxel+vehicle treated) resulted in significant reduction in hind paw withdrawal threshold to mechanical stimulus indicating development of paclitaxel-induced neuropathic pain. Initiation of treatment with the compound of Formula I in concurrence with paclitaxel injections (prophylactic treatment) inhibited the development of paclitaxel induced neuropathic pain dose dependently, of which the maximum effect was observed at the 3 mg/kg/day dose. Withdrawal of the compound of formula (I) treatment on day 15th post first paclitaxel injection did not cause any reversal of the drug's anti-allodynic effect until 7 days post last drug treatment (day 22 of study).

The day 16 score was reduced in the preventive model by >60% (p<0.001) at the 1 and 3 mg/kg dose levels and a similar reduction was observed in the therapeutic model. In the therapeutic treatment model, initiation of treatment with the compound of formula (I) at an oral dose of 3 mg/kg/day after establishment of paclitaxel induced neuropathic pain, resulted in reversal of neuropathic pain in time dependent manner, with the maximum effect recorded after the 5th daily dose with a sustained effect thereafter. Based on these findings it can be concluded that treatment with the compound of formula (I) is effective both as a prophylactic and therapeutic and has an anti-allodynic effect in paclitaxel induced neuropathic pain.

### Example 9: Efficacy of Compound of a Formula (I) in an Animal Model of Multiple Sclerosis

The oral treatment with a compound of formula (I) was conducted in an animal model of multiple sclerosis. EAE was induced in female Lewis rats with guinea pig MBP (25 μg/rat). Rats that developed EAE were divided into 3 groups (n=6) and compound of Formula I was administered as oral (1.3 mg/kg) or FTY720 (1 mg/kg) orally every day until day 26. Both the compound of Formula I and FTY720 showed similar efficacy in reducing clinical signs of disease, while the lymphopenia was less in animals treated with the compound of formula (I). Further, the lymphopenia observed in compound of formula (I) treated rats was short-term and quickly reversed whereas the lymphopenia observed in FTY720 treated rats was induced at a high level over a long-term period.

### Example 10: Efficacy of Compound of a Formula (I) in In-Vivo Inflammation

The efficacy of compound of formula (I) was determined in an animal model of dinitrofluorobenzene (DNFB)-induced delayed-type hypersensitivity (DTH), an inflammatory model. The animals (n=9) were treated with the vehicle or a compound of formula (I), receiving a twice daily dose of 3 mg/kg. The efficacy end points were measured as Ear thickness before challenge and 24 h after challenge. Ear weight was measured 24 h after challenge. At sacrifice, right ear samples were collected and used for tissue MPO activity. Administration of a compound of formula (I) significantly reduced ear thickness (˜70%, p<0.0001) and ear weight (˜50%, p<0.01) as well as MPO activity.

### Example 11: Efficacy of Compound of a Formula (I) in an Animal Model of Stroke

The compound of Formula I was assessed in an animal model of stroke, namely the middle cerebral artery occlusion (MCAO) model. The effect of a compound of Formula I on infarctions (TTC staining) and BBB leakage (Evan's blue extravasation) ischemia for 60 min and reperfusion for 72 h was assessed. The oral dose of 1, 3 and 5 mg/kg of a compound of formula (I) significantly reduced the infarct volume, infract area and sensory motor function and blood brain barrier leakage.

### Example 12: Efficacy of Compound of a Formula (I) in an Animal Model of Sepsis

Sepsis was induced in female Sprague-Dawley rats by LPS (5 mg/kg) administration and treated orally with compound of Formula I (3 mg/Kg) 1 h after and every 24 h thereafter to determine the effect of a compound of formula (I) in this animal model of sepsis (LPS mediated systemic inflammation). Animals were sacrificed at 24 and 72h post LPS treatment. There was positive impact on the body temperature and organ histopathology.

### Example 13: Efficacy of Compound of a Formula (I) in an Animal Model of Ulcerative Colitis

The effect of a compound of formula (I) was assessed in an animal model of Ulcerative colitis. 24 Balb/C Mice were divided into two groups; control and treatment (n=6). Acute colitis was induced in all groups by adding 2.5% w/v Dextran Sulphate Sodium (DSS) treatment in drinking water for 5-7 days. A compound of Formula I was administered at dose of 3 mg/kg body weight by oral route as a repeated dose for 3 or 6 days, consecutively. The daily administration of a compound of Formula I resulted in improvement of DSS-induced colitis, and significantly reduced the microscopic changes observed in colon. Gross pathological observations revealed that day 3 group animals were slightly emaciated and the anal areas were soiled with blood in both groups. During day 6 these observations were reduced to 16.66% in control and 0.00% in a compound of Formula I group.

The length of colon measured revealed that the mean colon length of control groups was short than the mean colon length of treatment groups. The mean colon lengths were 9.67 cm, 9.88 cm, 11.20 cm and 13.02 cm in day 3 control group, day 6 control group, day 3 a compound of Formula I treated group and day 6 a compound of Formula I treated group respectively. Histopathological evaluation of colon indicated the control animal displayed a severe increase in thickness of mucosa of 2/6 animals, minimal in ⅙ animals and a mild increase in 3/6 animals. In case of a compound of Formula I treated animals, the severity was reduced and the incidence of increase in thickness of mucosa was mild in 3/6 animals and minimal in 2/6 animals. The mucus secreting goblet cells in control animals were absent in 3/6 animals, whereas the number of mucus secreting goblet cells was increased moderately ( 4/6) in a compound of Formula I treated animals. The severity of presence of haemorrhages with desquamated cells seen in lumen of colon of control animals was reduced in a compound of Formula (I) treated animals. Histopathological evaluation on day 6 revealed reduction in infiltration of MNC and was minimal and focal in ⅚ animals and moderate in ⅙ control animals. In the compound of formula (I) treated group animals, it was minimal in 2/6 animals and mild in ⅙ animals. Thickness of mucosa was also markedly reduced in compound of Formula I treated animals compared to control animals. There was moderate ( 4/6 animals) to minimal (⅙ animals) increase in mucus secreting goblet cells in a compound of Formula I treated animals as compared with those of controls.

### Example 14: Efficacy of Compound of a Formula (I) in an Animal Model of Epilepsy

In an animal model of epilepsy, Sprague Dawley rats (n=6) were treated with the compound of Formula I or vehicle, dosed 1 hour prior to kainic acid administration (10 mg/kg, IP). One-hour post kainic acid administration, the rats were observed for behavioural changes (grooming, rearing, hind limb scratching, urination, defecation, wet dog shakes, jaw movements, salivation, head nodding), incidence and latency of convulsions and mortality until 1 hour. There was significant reduction in seizures (70%, p<0.005) in the compound of formula (I) treated group. The changes observed in the hippocampal region of control animals included, minimal to mild neuronal cell death which included vaculations in neuronal cells particularly at C3 and C1 regions proving the compound of Formula I is neuroprotective.

### Example 15: Efficacy of Compound of a Formula (I) in Arthritis Patients with Pain

Fifteen arthritis patients who were experiencing pain were treated with the compound of Formula I@60 mg daily dosing as a topical treatment for seven consecutive days. The drug appeared to be safe and well tolerated and there were no serious or significant adverse events (AEs) observed (all AEs were in the mild to moderate category). The subjects blood parameters, urine alysis, vital signs, physical examination, ECG were normal. In all participants treated with compound of formula (I) (n=12) there was an overall positive response to patient global assessment of response to therapy (PGART) scores observed at Day 3 (41.7% participants) and at Day 7 (58.3% participants). In participants with osteoarthritis a positive PGART response was observed by Day 3 (28.6% participants) and at Day 7 (57.1% participants). The placebo participants (n=3) did not received a positive response.

The numerical rating scale for pain scoring (NRS) in a compound of Formula I treated group (n=12) reduced from 7.1±1.38 (Day 1; baseline) to 5.8±2.12 (Day 3) and 4.8±2.72 (Day 7). The mean NRS score change from baseline was −1.3±1.54 (Day 3) and −2.3±2.06 (Day 7) points in arthritis participants treated with compound of Formula I. In osteoarthritis participants, the mean change in pain score from Day 1 to Day 7 was of −1.9±1.46 and −1.9±1.57 for pain felt and worst pain felt in 24 hours, respectively. This reduction in NRS score in OA participant was found to be significant (p=0.0153 and 0.0205, respectively). The topical application of a compound of Formula I in participants with arthritis for 7 days appeared to be safe, efficacious, and well tolerated and resulted in significant and fast reductions in the pain scores over time. There was no systemic change in the absolute lymphocyte count. The systemic exposure on day 7 ranged from 0.42 ng/mL to 2.44 ng/mL (average 0.79 ng/mL).

### Example 16: Efficacy of Compound of a Formula (I) in Psoriasis Patients

Twelve psoriasis patients were treated with the compound of Formula I@30 mg daily dosing under occlusion as topical treatment for 28 consecutive days. A significant reduction compared to baseline in the overall local psoriasis severity index (LPSI) score were noted in a compound of formula (I) group at each visit from Day 7 through to Day 28. The LPSI score reduced from 5.8 (day 1) to −2.1 (day 28) (p<0.0016), and no significant change was observed in the placebo group. Some subjects in the compound of Formula I group showed a reduction in plaque area, as assessed using image analysis of clinical photographs. The systemic exposure after topical applications was recorded below the limit of quantification (BLQ) <0.400 ng/mL. There was no systemic change in the absolute lymphocyte count. The study drug appears to be well tolerated as evidenced by adverse events, laboratory blood parameters, urinalysis, vital signs, physical examination and ECG.

### Example 17: Efficacy of Compound of a Formula (I) in Atopic Dermatitis Patients

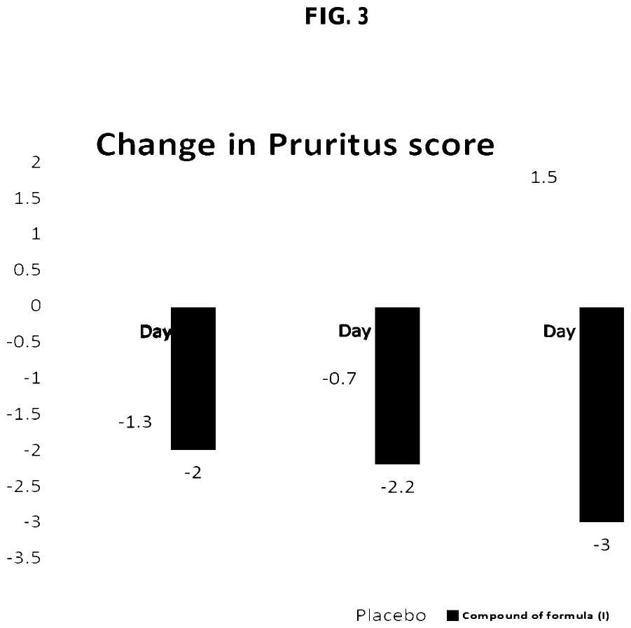

Atopic dermatitis patients were treated with the compound of Formula I@60 mg daily dosing as topical treatment for 28 consecutive days. A reduction compared to baseline in the overall eczema area & severity index (EASI) score and pruritus score (FIG. 3) were noted with a compound of formula (I). There was no systemic change in the absolute lymphocyte count. The compound of formula I appears to be well tolerated as evidenced by adverse events, laboratory blood parameters, urinalysis, vital signs, physical examination and ECG. The systemic exposure by week 3 was ≤1 to 2.5 ng/mL with no lymphopenia event occurred.

### Example 18: Efficacy of Compound of a Formula (I) in Healthy Human Subjects

Human subjects were treated in cohorts of different dose levels (each cohort=8 human subjects) with a compound of formula (I)@0.5 mg, 2 mg, 6 mg and 12 mg single oral dose. The drug was safe with no bradycardia events reported after 72 hours of clinical monitoring and ECGs (12 mg dose shown in FIG. 4 for one of the cohorts).

One cohort (8 human subjects) was treated daily with 8 mg oral dosing for 7 consecutive days. The systemic exposure reached to mean value of 53.3 ng/mL and only mild lymphopenia was observed (Table 1).

TABLE 1
Effects of compound of formula (1) on absolute lymphocyte count (ALC), CD3, CD4
and CD8 counts in healthy human subjects.

| Subject No | ALC Change (%) | CD3 Change (%) | CD4 Change (%) | CD8 Change (%) |
|---|---|---|---|---|
| 101 | +40 | −12.58 | −10.91 | −13.79 |
| 102 | −22.58 | −5.77 | −8.21 | 0 |
| 103 | −15.56 | −12.36 | −25.3 | −7.06 |
| 104 | −20 | −11.86 | −7.87 | −11.86 |
| 109 | −33.33 | −18.75 | −22.41 | −19.05 |
| 110 | −27.66 | −30.32 | −33.62 | −27.87 |
| 111 | −35.71 | −30.58 | −32 | −31.74 |
| 112 | 0 | −0.6 | −5.2 | −1.67 |

### Example 19: Systemic Exposure of Compound of a Formula (I) in Human Subjects after Topical Application

The body surface area of an adult is around 16,000 cm² to 18,000 cm² and the skin surface area varies from 10,000 cm² to 20,000 cm² (https://hypertextbook.com/facts/2001/IgorFridman.shtml, and references cited within). Human subjects received a topical application of 1 g of 3% by weight formulation per 150 cm² of body surface area (over the skin) twice daily for 21 days. The resulting systemic exposure was approximately 2.2 ng/ml. In another cohort, the systemic exposure via local application of 2 g of 3% or 6% by weight formulation with the amount of compound of formula (I) as 60 mg or 120 mg over the joint area for one week did not exceed 2.5 ng/ml.

### Example 20: Preparation of Tablet Formulation of a Compound Formula (I)

Lactose monohydrate was passed through U.S. Mesh size #40 and collected in a clean sterile poly lined container, maize starch was then added and passed through U.S. Mesh size #100. Following this, microcrystalline cellulose was passed through U.S. Mesh size #40 mesh and the composition was dry-mixed. Povidone solution was used as a binder solution (polyvinylpyrrolidone K 30) and subsequently filtered through the U.S. Mesh size #100. The granules were dried in a hot oven (55 to 60 deg. C) for approximately 25 to 30 minutes or until the granules had dried to the desired levels. Then granules were sifted through U.S. Mesh size #20 and collected in clean and sterile polylined containers. Colloidal silicon dioxide was added and the mixture subsequently sifted through U.S. Mesh size #40, and mixed for 3 minutes. Then, magnesium stearate was sifted through U.S. Mesh size #60, and mixed for 3 minutes. The mixture was then compressed into the desired tablet formulation. Table 2 lists the ingredients used in the above described process.

TABLE 2

| | Pharmacopoeia | Standard | Standard |
|---|---|---|---|
| Ingredients | grade | quantity/mg | quantity/kg |
| Compound of Formula 1 | IH | 2.00 | 0.0200 |
| Lactose monohydrate | BP | 28.250 | 0.2825 |
| Maize starch | BP | 21.600 | 0.2160 |
| Microcrystalline cellulose | BP | 28.250 | 0.2825 |
| Sodium starch glycolate | BP | 2.700 | 0.0270 |
| Povidone (PVP K 30) | BP | 3.600 | 0.0360 |
| Purified water* | IH | q.s. | 0.2800 |
| Colloidal silicon dioxide | USP | 0.540 | 0.0054 |
| Magnesium Stearate | BP | 0.360 | 0.0036 |

*Not present in the final product

### Example 21: 3% w/w Ointment Composition of S1P1 Agonist of Formula (I), Free Base, for Topical Use

A mixture of Vaseline (30.8 g) and Gelucire 50/13 pellets (4 g) was melted and stirred at ˜70° C. until homogenous (˜15 min). A solution of compound of formula (I), free base, (1.2 g) in anhydrous DMSO (4 ml) was added to the mixture with vigorous stirring. The mixture was allowed to cool to room temperature and the resultant ointment (40 g), contained 3% (w/w) of free base of a compound of formula (I).

### Example 22: 3% w/w Gel Composition HCl Salt of Formula (I), for Topical Use

A mixture of H₂O (4.85 g) and propylene glycol (4.85 g) and cellosize PCG 10 (0.3 g) was prepared. The mixture was allowed to stir overnight at room temperature to give a transparent viscous gel (10 g). This gel (6 g) was mixed with EtOH (4 g) and the resulting mixture was stirred at ˜70° C. for 2 h. To it a hydrochloride salt of a compound of formula (I) (0.45 g), dissolved in anhydrous DMSO (3 g) was added at once and EtOH was added to give a final mass of 15 g. The resulting mixture was stirred for 1 hour at ˜70° C., to give a transparent colourless gel with excellent stability and spread ability.

### Example 23: 3% w/w Gel Composition of Formula (I), Mesylate Salt, for Topical Use

When the hydrochloride salt of a compound of formula (I) of Example 7 was substituted for the mesylate salt of a compound of formula (I), an identical process gave the title composition.

### Example 24: 3% Liquid Composition of Formula (I), Mesylate Salt, for Topical Use

A mesylate salt of the compound of formula (I) (0.3 g) was dissolved in 50% aqueous DMSO (4 g) and this was diluted to 10 g with EtOH, to give the title formulation as a colourless liquid (10 g).

### Example 25: 1% Liquid Composition of Formula (I), HCl Salt, with Polyvinyl Pyrrolidone (PVP) for Topical Use

A HCl salt of a compound of formula (I) (0.05 g) was dissolved in 80% aqueous EtOH (4.45 ml). To it, polyvinyl PVP (0.5 g) was added and the mixture was stirred until completely homogenous (˜1 h) at room temperature to give a stable colourless solution, which formed a film after application to the skin.

### Example 26: 0.5% Sterile Aqueous Solution of Formula (I), Mesylate Salt, for Injection/Liquid Oral Formulation/Drops for Eye and Ear Administration

To a sterile container with a mesylate salt of a compound of formula (I), (0.005 g), sterile isotonic solution was added (1 ml) via syringe and the resulting mixture was stirred at room temperature by shaking until homogenous, which may be used for injection, eye or ear drops or orally.

### Example 27: Topical Patch Formulation of Formula (I)

A compound of formula (I) and other ingredients including solubility enhancers or permeation enhancers such as but not limited to DMSO, polyvinyl pyrrolidones (PVPs), glycyryl laurates, lauryl lactate, aerosol, eudragit may be dissolved in solvent (ethanol, propanol, isopropanol). An adhesive is added and mixed until homogenous. The homogenous slurry at optimal temperature may be casted onto a release layer (silicone or fluoropolymer coated polyester film and dried.

### Example 28: 3% w/w Ointment Composition of S1P1 Agonist of Formula (I), Free Base, in Combination with 1% Nicotinamide and 2% Vitamin E for Topical Use

A compound of formula (I) as a free base, (0.6 g), nicotinamide (0.2 g),vitamin E (d isomer; 0.4 g), Gelucire 50/13 pellets (2 g), polysorb 20 (0.6 g) in anhydrous DMSO (2 ml) were stirred at ˜55° C. until homogenous (˜30 min). Melted Vaseline was added to make a final weight of 20 g. This was vigorously stirred for 15 min at ˜50° C., cooled to room temperature to give an off-white ointment.

### Example 29: 3% w/w Ointment Composition of S1P1 Agonist of Formula (I), Free Base, and 0.05% w/w of Betamethasone for Topical Use

A mixture of Vaseline (30.78 g) and Gelucire 50/13 pellets (4 g) was melted and stirred at ˜70° C. until homogenous (˜15 min). To it a solution of compound of formula (I), free base, (1.2 g) and betamethasone (0.02 g) in anhydrous DMSO (4 g) was added with vigorous stirring. The mixture was allowed to cool to room temperature to give cloudy ointment (40 g), containing 3% (w/w) of free base of a compound of formula (I) and 0.05% of betamethasone.

### Example 30: 2% w/w Gel Composition HCl Salt of Formula (I) and 1% Diclofenac for Topical Use

A solution of solution of H₂O (4.85 g) and propylene glycol (4.85 g) and cellosize PCG 10 (0.3 g) was prepared. The mixture was allowed to stir overnight at room temperature to give a transparent viscous gel (10 g). This gel (6 g) was mixed with EtOH (3.9 g) and the resulting mixture was stirred at ˜70° C. for 2 h. To it a mixture of hydrochloride salts of a compound of formula (I) (0.3 g) and diclofenac (0.15 g), dissolved in anhydrous DMSO (4.5 g) was added at once and EtOH was added to give a final mass of 15 g. The resulting mixture was stirred for 1 hour at ˜70° C., to give the titled product as a transparent colourless gel with excellent stability and spreadability.

### Example 31: Use of a Topical Formulation of a Compound of Formula (I) in Wound Patients

A 68-year old male patient presented with a second degree burn wound on the inner surface of his middle finger of his left hand, suffering from swelling, blistering and pain at the site of injury. A topical gel formulation of compound of formula (I) was applied topically to the site of injury. After 10 minutes, the swelling and blistering had visibly reduced dramatically and the patient reported significant pain relief.

A 49-year old male patient presented with a scratch wounds on hands while cleaning gutters. These become inflamed and painful in 3 hours. The ointment formulation of compound of formula (I) was applied topically to the site of injury, the swelling and pain reduced dramatically and the patient reported significant pain relief. No adverse side effects were reported in either case.

### Example 32: Alternative Formulations of Compounds of Formula I

The compound of formula I is soluble in water as salt form such as HCl, mesylate salt giving a stable clear solution. The compound of formula I free base (free amine form) is insoluble in water. To solubilize the free base in water the novel technology AvignaSOL (N,N-Dimethyl hexanamide; patent number: U.S. Pat. No. 9,186,338B2) and its higher derivatives such as octinamide, decamide which improve the absorption of various poorly soluble drugs was used. AvignaSOL as 1-70% ww % assisted the solubilization of free base as 1 gm/20 mL of water. The solubility of free base in water assisted its direct use in various formulation such as creams, gels, solutions which together with the permeation enhancing effect will be harnessed to improve skin penetration and/or bioavailability and in altering the pharmacokinetics and pharmacodynamics profile of free base.

The AvignaSOL having a log P of ˜1.6 hinder the release by increasing the hydrophobicity of the delivery system. The solubilized compound of Formula I-AvignaSOL does not precipitate in phosphate buffer pH 6.8. Hence AvignaSOL can be used to increase the bioavalibility of compound of formula I but not limited via intestinal and/or via dermal. The combination of compound of formula I with other ingredients including sustained release enhancer with or without AvignaSOL were prepared as novel formulation techniques to improve and/or alter the pharmacokinetics and pharmacodynamics profile of compound of Formula I.

To improve the pharmacokinetics profile such as half-life (T½) and Cmax the compound of formula I was formulated in various ingredients and solvents listed in Table 6:

TABLE 6
Compound of formula I formulations

| | | | Range |
|---|---|---|---|
| S.No | Ingredient | Function | % w/w |
| 1 | AvignaSOL and higher derivatives octa and deca form | Solubulizer | 0-40% |
| 2 | Lactose | Diluent | 0-30% |
| 3 | Dicalcium phosphate | Diluent | 0-30% |
| 4 | Isomalt | Diluent | 0-30% |
| 5 | Microcrystalline cellulose | Diluent | 0-30% |
| 6 | Partially Pregelatinized Maize starch | Binder | 0-20% |
| 7 | Hydroxy propyl cellulose | Binder | 0 to 40% |
| 8 | Polyvinyl pyrrolidone | Binder | 0-15% |
| 9 | Ethyl cellulose | Binder/Release retardant | 0-20 % |
| 10 | Hydroxy propyl methyl cellulose K100 M | Release retardant | 0-30% |
| 11 | Hydroxy propyl methyl cellulose K200 M | Release retardant | 0-30% |
| 12 | Hydroxy propyl methyl cellulose K15M | Release retardant | 0-30% |
| 13 | Hydroxy propyl methyl cellulose K4 M | Release retardant | 0-30% |
| 14 | Poly ox N-750 | Release retardant | 0-30% |
| 15 | Polyox WSR-301 | Release retardant | 0-30% |
| 16 | Poly ox WSR-303 | Release retardant | 0-30% |
| 17 | Poly ox WSR-205 | Release retardant | 0-30% |
| 18 | PolyoxN1105 | Release retardant | 0-30% |
| 19 | Polyox WSR-N-12K | Release retardant | 0-30% |
| 20 | Polyox WSRN60K | Release retardant | 0-30% |
| 21 | EudragitRL 100 | Release retardant | 0-30% |
| 22 | EudragitRS 100 | Release retardant | 0-30% |
| 23 | Glyceryl behenate | Binder/ Release retardant | 0-30% |
| 24 | Caranauba wax | Release retardant | 0-30% |
| 25 | Xanthan gum | Release retardant | 0-30% |
| 26 | Colloidal silicon dioxide | Flow improver | 0-1% |
| 27 | Magnesium stearate | Lubricant | 0-3 % |
| 28 | Sodium stearyl fumarate | Lubricant | 0-3% |
| 29 | Polyvinyl alcohol | Tablet coating agent | 0 to 20% |
| 30 | Hypromellose | Tablet coating sustained | 0 to 20% |
| | | release agent | |
| 31 | Polyethylene glycol | Tablet coating agent | 0 to 10% |
| 32 | Titanium dioxide | Tablet coating agent | 0 to 10% |
| 33 | Talc | Tablet coating agent | 0 to 10% |
| 34 | hydroxy ethylcellulose | In gel and tablet coating | 0-20% |

Skin permeation enhancers for topical formulations

| | | | |
|---|---|---|---|
| 1 | AvignaSOL | NA | 0 to 40% |
| 2 | Isopropyl myristate | NA | 0 to 20% |
| 3 | Dimethyl isosorbide. | NA | 0 to 20 % |
| 4 | Propylene Glycol | NA | 0 to 40% |
| 5 | Glycerol | NA | 0 to 40% |
| 6 | Glycofural | NA | 0 to 40% |

Other ingredient

| | | |
|---|---|---|
| 11 | Tween 80 | 0-20% |
| 12 | Glyceryl monostearate | 0-20% |
| 13 | Glyceryl distearate | 0-20% |
| 14 | Dimethyl isosorbide | 0-20% |
| 15 | Stearic acid | 0-20% |
| 16 | Cetyl alcohol | 0-15% |
| 17 | Stearyl alcohol | 0-15% |
| 18 | Purified water | 0-98% |
| 19 | Cetearyl alcohol | 0-15% |
| 23 | Isopropyl myristate | 0-20% |
| 24 | Petroleum jelly | 0-95% |
| 25 | Paraffin light oil | 0-95% |
| 26 | SPAN 80 | 0-20% |
| 27 | SPAN 60 | 0-20% |
| 28 | Decyl glycoside | 0-20% |

i) Preparation of ointment: White soft paraffin, cetyl alcohol, glyceryl monostearate and light liquid paraffin was heated to 70 to 75° C. with mechanical stirring for 15 minutes when a clear solution (solution A) was obtained. Separately a clear solution (solution B) of propylene glycol, compound of formula I and stearic acid was prepared by heating with stirring for 15 minutes at 90 to 95° C. with magnetic stirrer. This solution B was added to solution A (maintained at 90 to 95° C.) with stirring by a mechanical stirrer. This mixture was then stirred at 380 rpm at 75 to 85° C. for 15 minutes. Then the mixture was cooled with stirring by mechanical stirrer at 380 rpm at 40 to 45° C. for 30 minutes and then at 300 rpm at 25 to 30° C. (ambient conditions) for additional 60 minutes to give a homogenous off-white ointment.

| Ingredients | % w/w | Range % w/w |
|---|---|---|
| Compound of formula I | 3.00 | 1 to 6 |
| Propylene glycol | 17.00 | 5 to 20 |
| White soft paraffin | 48.70 | 20 to 80 |
| (petroleum jelly) | | |
| Cetyl alcohol | 5.00 | 2 to 7 |
| Stearic acid | 1.80 | 0.5 to 4 |
| Glyceryl mono stearate | 6.00 | 2 to 8 |
| Light liquid Paraffin | 18.50 | 7 to 35 |
| Total | 100.00 | (petroleum jelly |
| | | proportion was adjusted) |

ii) Preparation of gel or spray: To the mixture of propylene glycol, glycerol, water with or without the dimethyl isosorbide was added the compound of formula I and stirred (60 to 70° C.) till the solution is clear. To this was added the hydroxy ethyl cellulose or HPMCK100 or hydroxy propyl cellulose or hydroxy methyl cellulose (Cellosize) and the content was stirred at 55° C. for 1 hr to give a clear gel or solution.

| Ingredients | % w/w | Range %w/w |
|---|---|---|
| Compound of formula I | 3.00 | 1 to 6 |
| Propylene glycol | 15.00 | 5 to 20 |
| Glycerol | 5.00 | 2 to 20 |
| Dimethyl isosorbide | 1.50 | 0 to 5 |
| Water | 74.30 | 40 to 90 |
| Hydroxy propyl cellulose | 0.1 for spray and 1.20 | 0.1 to 4 |
| Or Hydroxy ethyl cellulose | for gel | |
| Total | 100.00 | (water proportion |
| | | was adjusted) |

iii) Preparation of extended-release granules: Compound of formula I was dissolved in AvignaSOL by bath sonication and then triturated with mixture of Isomalt and MCC PH101 in a mortar. Further other ingredients: ethyl cellulose, HPMC K100 M and Pregelatinized starch were added by trituration in a mortar and then co-sifting through #30 mesh-3 times. Then the solution of 5 ml of IPA: Water (80:20) containing ethyl cellulose was added to granulate the mixture. In another experiment the compound of formula I was blended with Isomalt, MCCPH101, ethyl cellulose 7 cps, HPMC K100 M and Pregelatinized starch and then co-sifting through #30 mesh-3 times. Then the solution of 5 ml of IPA: Water (80:20) containing ethyl cellulose was added to granulate the mixture. The wet granules were sieved through #20 mesh and dried in a hot air oven at 65° C. for 2.5 hrs. The granules passing through #20 mesh and retained on #60 mesh was used for further dissolution study at 37 to 40° C., which exhibited an in-vitro release as follows: 1 hr (27%) and 19 hrs (71.4%).

### Example 33: The Anti-Mould (Antifungal) Activity of Compound of Formula I

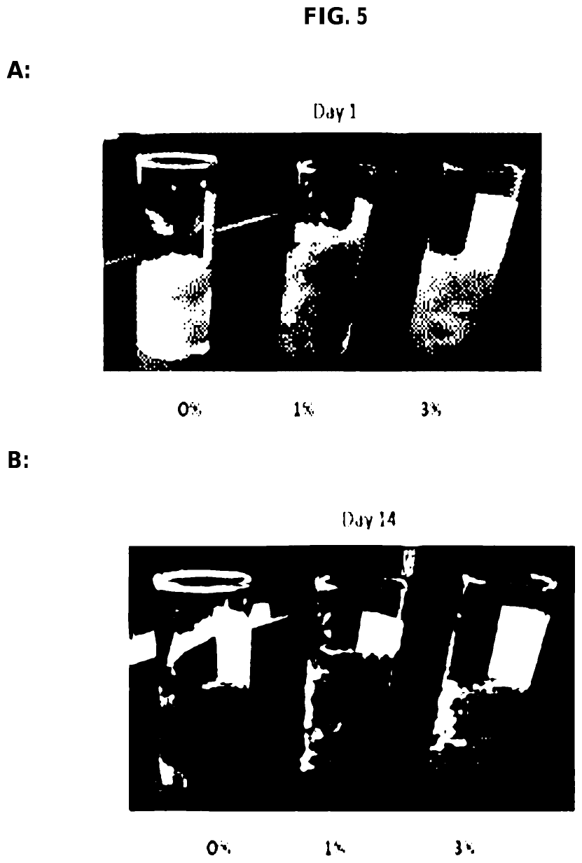

The compound of formula I solution as 0% ww, 1% ww and 3% ww as I mL water solution was added to a glass jar (5 mL) containing the fresh wheat flour bread (1 gm). The content was mixed well in order to wet the bread with solution. The content was kept in open glass cylinder for 14 days at room temperature and light (normal conditions) and the photos of Day 1 and Day 14 were taken (FIGS. 5A and B respectively).

The anti-mould activity of the compound of formula I was demonstrated by the jars containing compound of formula I preventing mould growth at day 14, as seen by the black colouring in the 0% jar. The mould is common in many skin indications and other human, animal and plant diseases. For example, Rhizopus stolonifera.

### Example 34: Topical Efficacy of Compound of Formula I for Pain in Freund's Adjuvant and Deep Incision Wound in Rat

The total 24 female Sprague Dawley Rats were assigned to two test and two control groups having 6 animals per group. Animals from G1 and G2 received CFA on Day 1 by intraplanter route followed application of test item (0 and 3% respectively), onto injection site from Day 1 to Day 7. Group G3 and G4 animals received deep incision through the skin and fascia of the plantar foot followed by application of test item (0 and 3% respectively), onto the incision site from Day 1 to Day 7. Clinical signs, body weight and pain assessment parameters like Von Frey estimation and FOB was evaluated and the pain reduction results at Day 7 are summarized in Table 7. Compound of formula I treatment inhibited the CFA induced and incision wound (neuropathic) pain in rats and has anti allodynic effect recorded at day 7 in CFA (complete pain relief) and deep incision induced neuropathic pain (67% relief).

TABLE 7
Pain reduction results at Day 7

| CFA mediated pain | | Incision mediated pain | |
|---|---|---|---|
| Placebo (G1) | 3% CFI (G2) | Placebo (G3) | 3% CFI (G4) |
| 0.17 | 0.00 | 0.33 | 0.11 |

### Example 35: In Vitro Efficacy of Compound of Formula I in Acne Bacteria Induced Inflammation

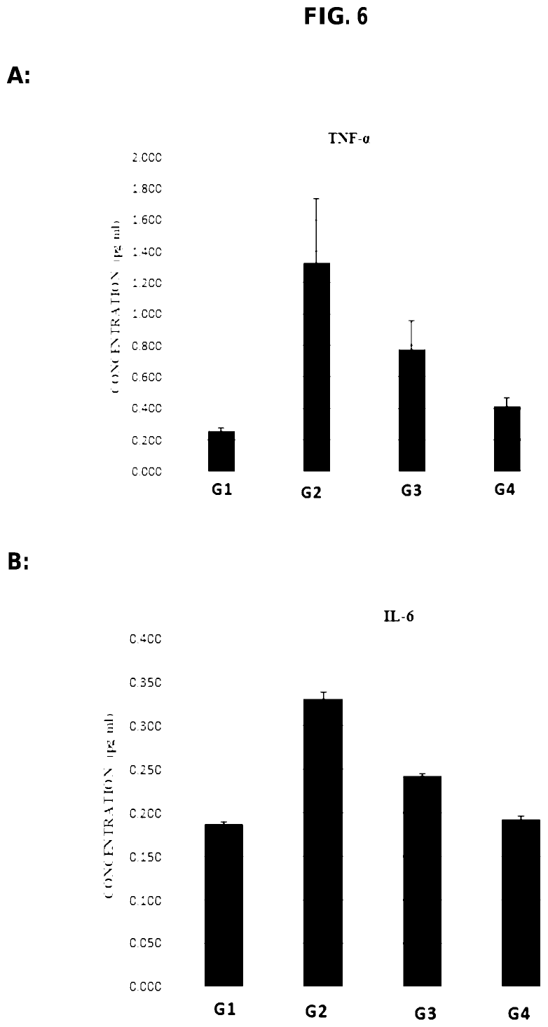

RAW 264.7 cell line (monocyte cells) challenged with the heat-killed Propionibacterium acne (P. acne) and incubated for 1 hour and were divided into four groups as G1=not challenged with P. acne, G2=challenged with P. acne but untreated, G3=challenged with P. acne and treated with 1 μM of compound of formula I, G3=challenged with P. acne and treated with 3 μM of compound of formula I. The ELISA method was used to analyze the effect of compound of formula I on the secretion of proinflammatory cytokines the TNFα and IL6. Compound of formula I inhibits the cytokines in P. acne challenged monocyte cells. The results are shown in below FIG. 6. The compound of formula I inhibit the secretion of TNFα@1 μM=˜42% and @3 μM=˜69% and IL6@1 μM=˜27% and @3 μM=˜42% compared to untreated in P. acne challenged cell line.

It is to be understood that while the present disclosure has been described in conjunction with the specific embodiments thereof, the foregoing description is intended to illustrate and not limit the scope of the disclosure. Other aspects, advantages and modifications will be apparent to those skilled in the art to which the disclosure pertains. Therefore, the following examples are put forth so as to provide those skilled in the art with a complete disclosure and description of how to make and use the disclosed compositions and are not intended to limit the scope of the disclosure.

All documents cited are herein fully incorporated by reference for all jurisdictions in which such incorporation is permitted and to the extent such disclosure is consistent with the description of the present disclosure.

1. A method of treating or preventing a disease or disorder by administering to a human subject in need thereof a medicament comprising an S1P receptor modulator, whereby said medicament decreases the heart rate of the subject by about 5 beats/min or less daily, or about 4 beats/min or less daily, or about 3 beats/min or less daily, or about 2 beats/min or less daily;
wherein the disease or disorder is selected from the group consisting of pruritis, pain, multiple sclerosis, ulcerative colitis, psoriasis, dermatitis and acne;
wherein the S1P receptor modulator is administered at an initial daily dosage which is substantially the same as the standard daily therapeutic dosage;
wherein the level of lymphopenia is ≤70%; and
wherein the S1P receptor modulator is a compound of formula (I): 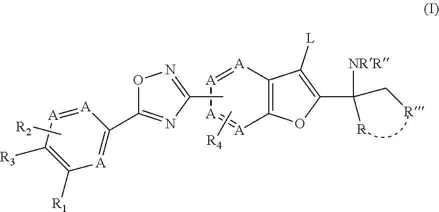
wherein R₁ is selected from the group consisting of hydrogen, deuterium, halogen, CN, CF₃, —COOH, amide, sulphonamide, alkoxy, aryloxy, nitro, and a C₁₋₆ alkyl group, said alkyl group optionally comprising one or more of deuterium, O, S, NR′ (R′═H, alkyl, cycloalkyl), halogen, a carbon-carbon double bond, a carbon-carbon triple bond, a carbon-nitrogen double bond, a carbon-nitrogen triple bond, heterocycle, aryl, alkyl and cycloalkyl (C₃₋₇);
wherein R₂ is selected from the group consisting of hydrogen, deuterium, halogen, CN, CF₃, alkoxy, aryloxy, and a C₁₋₄ alkyl group, said alkyl group optionally comprising one or more of deuterium, O, S, NR′ (R′═H, alkyl, cycloalkyl), halogen, a carbon-carbon double bond, a carbon-carbon triple bond, a carbon-nitrogen double bond, a carbon-nitrogen triple bond, heterocycle, aryl, and C₃₋₇ cycloalkyl;
wherein R₃ is selected from the group consisting of hydrogen, deuterium, halogen, alkoxy, aryloxy, and a C₁₋₆ alkyl group, said alkyl group optionally comprising one or more of deuterium, O, S, NR′ (R′═H, alkyl, cycloalkyl), halogen, a carbon-carbon double bond, a carbon-carbon triple bond, a carbon-nitrogen double bond, a carbon-nitrogen triple bond, heterocycle, aryl, alkyl, and C₃₋₇ cycloalkyl; preferably R₃ is selected from the group consisting of Me, OMe, OEt, OPr, O-iPr, O-isobutyl, O-isopentyl, O-cyclopentyl, O-allyl, O-benzyl and 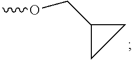
wherein R₄ is selected from the group consisting of hydrogen, deuterium, halogen, CN, CF₃, and a C₁₋₄ alkyl group, said alkyl group optionally comprising one or more of deuterium, O, S, NR′ (R′═H, alkyl, cycloalkyl), halogen, a carbon-carbon double bond, a carbon-carbon triple bond, a carbon-nitrogen double bond, a carbon-nitrogen triple bond, heterocycle, aryl, alkyl, and C₃₋₇ cycloalkyl;
wherein A, independently in each occurrence, represents a carbon or nitrogen atom with the proviso that a ring has no more than two nitrogen atoms;
wherein L is selected from the group consisting of hydrogen, deuterium, F, Cl, Br and a C₁₋₃ alkyl;
wherein R is selected from the group consisting of H, COOH, C₁₋₄ alkyl and C₁₋₄ hydroxy-alkyl;
wherein R′ and R″ are independently selected from H and C₁₋₄ alkyl;
wherein R′″ is selected from OH, —OPO₃H₂ and physiologically acceptable salts;
wherein [symbol] represents an optional bridging group;
or a pharmaceutically acceptable salt thereof.

2. A method according to claim 1, wherein the S1P receptor modulator is a compound of formula (II) 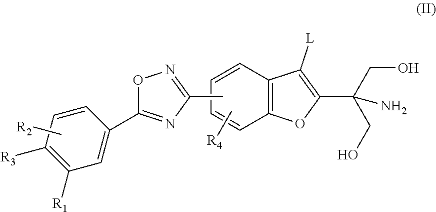
wherein R1 is selected from hydrogen, deuterium, halogen, CN, CF3, —COOH, amide, sulphonamide, alkoxy, aryloxy, nitro and an alkyl chain (C1-5), said alkyl chain optionally containing one or more of deuterium, O, S, NR′ (R′═H, alkyl, cycloalkyl), halogen, a multiple bond, heterocycle, aryl, and cycloalkyl (C3-7);
wherein R2 is selected from hydrogen, deuterium, halogen, CN, CF3, an alkyl chain (C1-4) said alkyl chain optionally containing one or more of deuterium, O, S, NR′ (R′═H, alkyl, cycloalkyl), halogen, a multiple bond, heterocycle, aryl, and cycloalkyl (C3-7);
wherein R3 is selected from hydrogen, deuterium, halogen, alkoxy, aryloxy, an alkyl chain (C1-7), said alkyl chain optionally containing one or more of deuterium, O, S, NR′ (R′═H, alkyl, cycloalkyl), halogen, a multiple bond, heterocycle, aryl, and cycloalkyl (C3-7);
wherein R4 is selected from hydrogen, deuterium, halogen, CN, CF3, an alkyl chain (C1-4), said alkyl chain optionally containing one or more of deuterium, O, S, NR′ (R′═H, alkyl, cycloalkyl), halogen, a multiple bond, heterocycle, aryl, and cycloalkyl (C3-7);
wherein L is selected from hydrogen, deuterium, F, Cl, Br and alkyl (C1-3).

3. A method according to claim 2, wherein the compound of formula (II) has
R1 selected from F, Cl, Br, CN, CF3, NO2, Me, OMe, OEt, OPr, O-iPr, O-isobutyl, O-isopentyl, O-cyclopentyl, O-allyl, O-benzyl and;
R2 selected from H, deuterium, F, Cl, Br, CN, CF3, NO2, Me, OMe, OEt, OPr, O-iPr, O-isobutyl, O-isopentyl, O-cyclopentyl, O-allyl, O-benzyl and;
R3 selected from H, deuterium, Pr, butyl, OMe, OEt, OPr, OiPr, O-isobutyl, O-isopentyl, O-butyl, O-pentyl, O-cyclopentyl, O-allyl, O-benzyl and;
R4 selected from H, deuterium, Me and Et; and
L selected from H, deuterium, Me and Cl.

4. A method according to claim 2, wherein the compound of formula (II) has
R1 selected from F, Cl, Br, CN, CF3, Me, NO2, OMe, OEt, OPr, O-iPr, O-isobutyl, O-isopentyl, O-cyclopentyl, O-allyl, O-benzyl and;
R2 is H;
R3 selected from H, deuterium, Pr, butyl, OMe, OEt, OPr, OiPr, O-isobutyl, O-isopentyl, O-butyl, O-pentyl, O-cyclopentyl, O-allyl, O-benzyl and;
R4 selected from H, deuterium, Me and Et; and
L is H.

5. A method according to claim 1, wherein the compound of formula (I) or formula (II) is selected from the group consisting of:

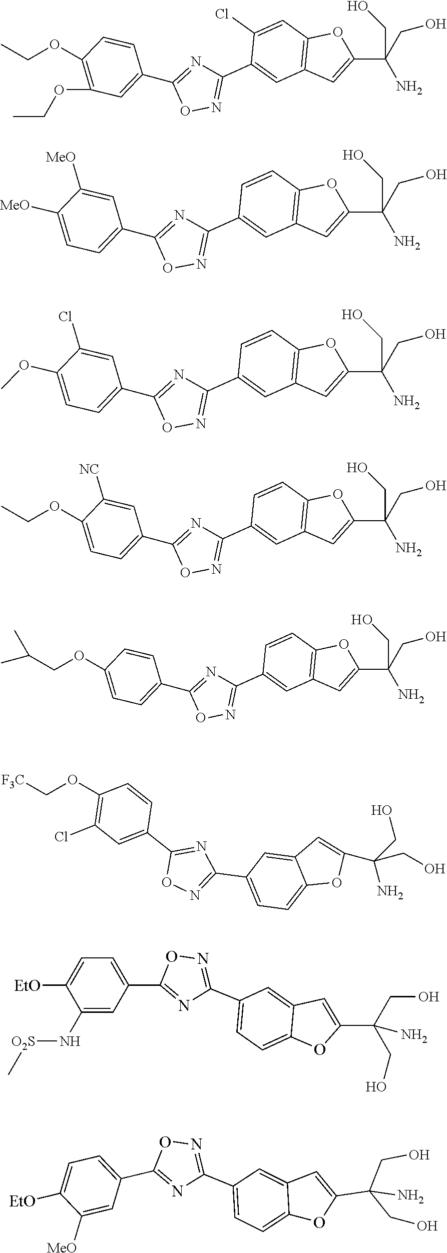

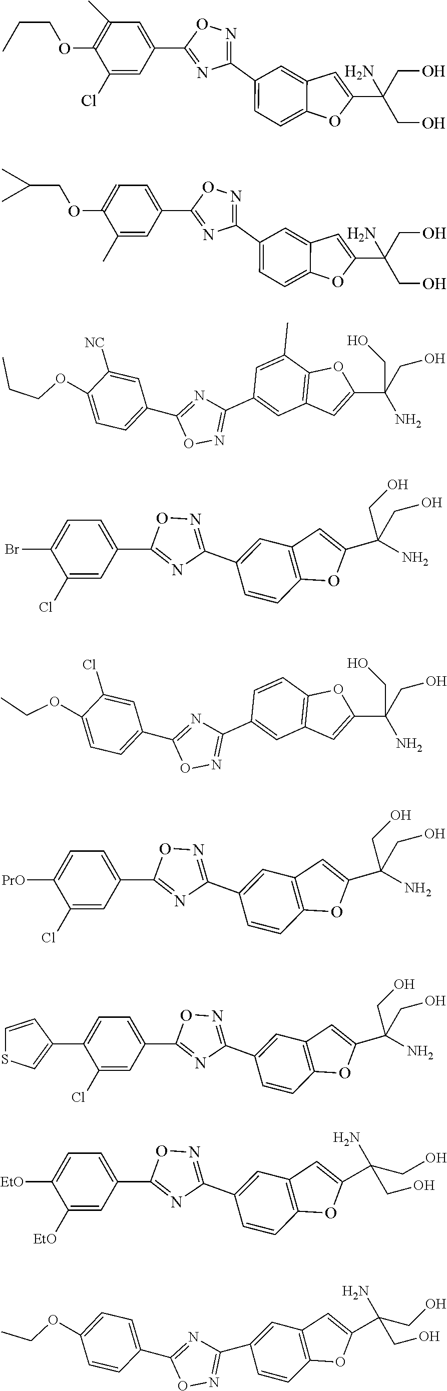

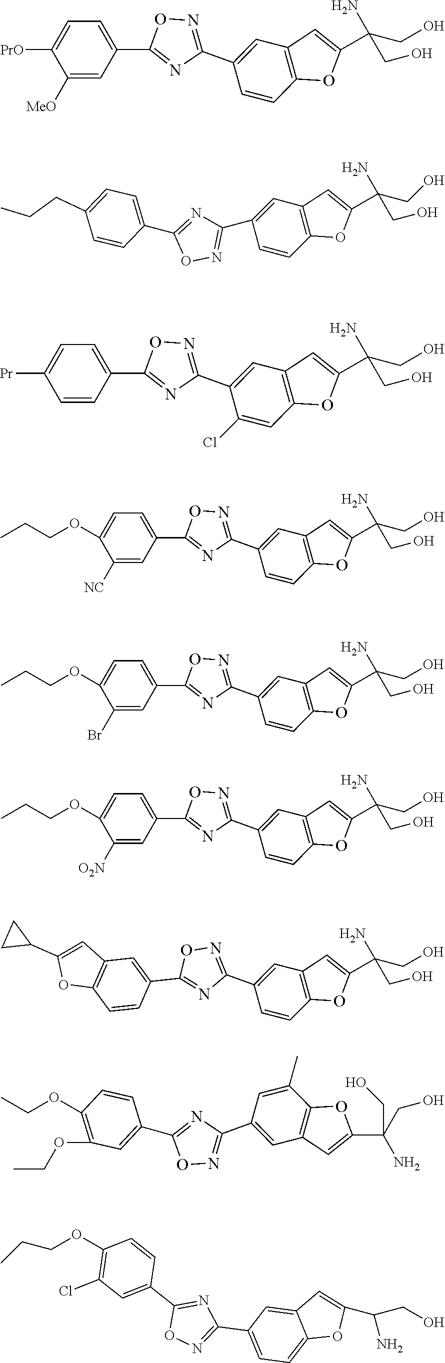

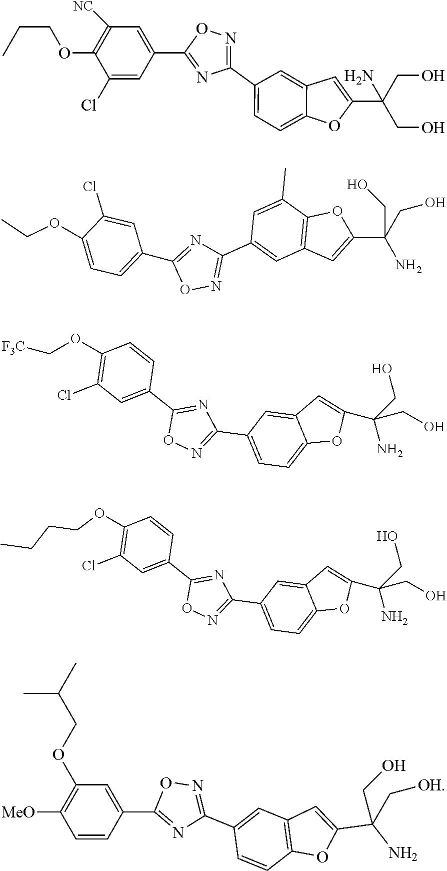

6. A method according to claim 1, wherein the difference between the initial daily dosage and the standard daily therapeutic dosage is less than 25%, or less than 15%, or less than 10%, or less than 5%.

7. A method according to claim 1, wherein the initial daily dosage is the same as the standard daily therapeutic dosage.

8. A method according to claim 1, wherein the standard daily therapeutic dosage of S1P receptor modulator is up to 70 mg.

9. A method according to claim 1, wherein the standard daily therapeutic dosage of S1P receptor modulator is up to 24 mg.

10. A method according to any claim 1, wherein the standard daily therapeutic dosage of S1P receptor modulator is between 0.5 mg and 12 mg.

11. A method according to claim 1, wherein the administration of the medicament does not cause a substantial decrease in heart rate.

12. A method according to claim 1, wherein the administration of the medicament does not cause bradycardia.

13. A method according to claim 1, wherein the level of lymphopenia is ≤25%.

14. A method according to claim 1, wherein the level of lymphopenia is ≤50%.

15. A method according to claim 1, wherein the medicament is administered to a subject who was previously under treatment with an alternate S1P1 modulator or agonist, and/or wherein said patient is currently undergoing discontinuation or cessation of treatment with an alternate S1P modulator or agonist.

16. A method according to claim 15, wherein said discontinuation or cessation of treatment is due to a bradycardia and/or lymphopenia event.

17. A method according to claim 1, wherein the medicament is an oral or injectable or systemic formulation, selected from a pill, a tablet, a capsule, a solution and a syrup.

18. A method according to claim 1, wherein the subject is susceptible to heart failure, arrhythmias, high grade atrio-ventricular blocks, sick sinus syndrome, has a history of Syncopal episodes or a combination thereof.

19. A method according to claim 18, wherein the subject is undergoing beta blocker or anti-arrhythmic treatment by receiving anti-arrthymic drugs.

20. A method according to claim 1, wherein the subject has undergone an interruption or treatment break from another S1P receptor modulator/agonist.

21. A method according to claim 20, wherein said treatment break is greater than 4, 6, 8, 10, 12, or 14 days.

22. A method according to, wherein the medicament is a slow release formulation, administered topically, by implantation or injection or via a medical device.

23. A method according to claim 1, wherein the medicament treats pain, selected from the group consisting of joint pain, arthritis pain, gout pain, back pain, muscle pain, neuropathy, neurologic pain, migraine, cancer pain, sports injury pain and wound pain.

24. A method according to claim 1, wherein the medicament comprises the S1P receptor modulator as a composition with another pharmaceutically active compound selected from immune suppressant/modulators agents, neuromodulators, anti-inflammatory agents, antipathogens, pain modulators, pruritus modulators, opioids, cannabinoids, antibacterial agents, antiviral agents and antifungal agents.

25. A method according to claim 1, wherein the medicament is in the form of a topical formulation selected from a solid, a patch, a powder, a liquid, a semisolid, an ointment, a gel, a spray, an aerosol, an inhaler and a lotion.

26. A method according to claim 1, wherein the medicament is administered topically, orally, transdermally, parenterally, intranasally, ocularly or rectally.

27. A method according to claim 1, wherein the medicament is applied topically.

28. A method according to claim 27, wherein the medicament comprises the S1P receptor modulator in an amount between 0.01% and 30% by weight.

29. A method according to claim 28, wherein the medicament comprises the S1P receptor modulator in an amount of about 3% by weight.

30. A method according to claim 27, wherein the medicament is applied to up to 1000 cm² of body surface area per 1 g of medicament, and wherein the standard daily therapeutic dosage of S1P receptor modulator is ≤3 g.

31. A method according to claim 30, wherein the standard daily therapeutic dosage of S1P receptor modulator is ≤1.5 g.
